SEQUENCE LISTING (15 sequences)

SEQ ID NO: 1 - DNA, 1809 bp, Macaca mulatta (cDNA for RhBchE)
atgcatagcaaagtcacaatcatatgcatcagattactcttttggtttcttttgctctgcatgcttattggaaagtcacatactgaagatgacatcgtaattgcaacaaagaatggaaaagtcagagggatgaacttaacagttcttggtggcacggtaacagcctttcttggaattccctatgcacagccacctcttggtagacttcgattcaaaaagccacagtctctgaccaagtggtctgatatttggaatgccacaaaatatgcaaattcttgctatcagaacatagatcaaagttttccaggcttccatggatcagagatgtggaacccaaacactgacctcagtgaagactgtttatatctaaatgtatggattccggcacctaaaccaaaaaatgctactgtaatgatatggatttatggtggtggttttcagactggaacatcatctttacatgtttatgatggcaagtttctggctcgagttgaaagagttattgtagtgtcaatgaactatagggtgggtgcccttggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccatccattgttcaccagagccattctacaaagtggatcctctaatgctccttgggcagtaacatctctttatgaagctaggaacagaacattgaccttggctaaattgactggttgctctagagataatgagactgaaatagtcaagtgccttagaaataaagatccccacgaaattcttctgaatgaagcatttgttgtcccctatgggactctcttgtcagtaaacttcggtccaaccatggatggtgattttctcactgaaatgccagacatattacttgaacttggacaatttaaaaaaacccagatattggtgggtgttaataaagatgaagggacagcttttttagtctatggtgctcctggcttcagcaaagataacgatagtatcataactagaaacgaatttcaggaaggtttaaaaatattttttccaggcgtgagtgagtttggaaaggaatccatcctttttcattacacagactgggtagatgatcagagacctgaaaactaccgtgaggcgttggatgatgttgttggggattataatatcatatgccctgccttggagtttaccaagaagttctcagaatggggaaataatgcctttttctactattttgaacaccgatcctccaaacttccgtggccagaatggatgggagtgatgcatggctatgaaattgaatttgtctttgggttacctctggaaagaagagttaattacacaaaagctgaggaaattttgagtagatccatagtgaaacggtgggcaaattttgcaaaatatgggaatccaaatgggactcataataatagcacaaaatggcctgtcttcaaaagcactgaacaaaaatatctaaccttgaatacagagtcatcaagaatattgactaaactacgtgctcagcaatgccgattctggacatcattttttccaaaagtcttggaaatgacaggaaatattgatgaagcagaatgggagtggaaagcaggattccatcgctggagcaattacatgatggactggaaaaatcaatttaacgattacactagcaagaaagaaagttgtgtgggtctctaa

SEQ ID NO: 2 - protein, 602 aa, Macaca mulatta
MHSKVTIICIRLLFWFLLLCMLIGKSHTEDDIVIATKNGKVRGMNLTVLGGTVTAFLGIPYAQPPLGRLRFKKPQSLTKWSDIWNATKYANSCYQNIDQSFPGFHGSEMWNPNTDLSEDCLYLNVWIPAPKPKNATVMIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHPLFTRAILQSGSSNAPWAVTSLYEARNRTLTLAKLTGCSRDNETEIVKCLRNKDPHEILLNEAFVVPYGTLLSVNFGPTMDGDFLTEMPDILLELGQFKKTQILVGVNKDEGTAFLVYGAPGFSKDNDSIITRNEFQEGLKIFFPGVSEFGKESILFHYTDWVDDQRPENYREALDDVVGDYNIICPALEFTKKFSEWGNNAFFYYFEHRSSKLPWPEWMGVMHGYEIEFVFGLPLERRVNYTKAEEILSRSIVKRWANFAKYGNPNGTHNNSTKWPVFKSTEQKYLTLNTESSRILTKLRAQQCRFWTSFFPKVLEMTGNIDEAEWEWKAGFHRWSNYMMDWKNQFNDYTSKKESCVGL

SEQ ID NO: 3 - protein, 602 aa, Homo sapiens
MHSKVTIICIRFLFWFLLLCMLIGKSHTEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 4 - protein, 581 aa, Rabbit
MVTRSSHTEDVIITTKNGRIRGINLPVFGGTVTAFLGIPYAQPPLGRLRFKKPQSLTKWSDIWNATKYANSCCQNIDQSFPGFHGSEMWNPNTDLSEDCLYLNVWIPTPKPKNATVMIWIYGGGFQTGTSSLQVYDGKFLTRVERVIVVSMNYRVGALGFLALPGNPEAPGNMGLFDQQLALQWVQKNIAAFGGNPKSVTLFGESAGAASVSLHLLSPRSHPLFTRAILQSGSSNAPWEVMSLHEARNRTLTLAKFVGCSTENETEIIKCLRNKDAQEILLNEVFVVPFDSLLSVNFGPTVDGDFLTDMPDTLLQLGQLKKTQILVGVNKDEGTAFLVYGAPGFSKDNTSIITRKEFQEGLKIFFPGVSEFGKESILFHYTDWVDEQRPENYREALDDVVGDYNFICPALEFTKKFSEWGNNAFFYYFEHRSSKLPWPEWMGVMHGYEIEFVFGLPLERRVNYTKAEEILSRSIMKRWANFAKYGNPNGTQNNSTRWPVFKSTEQKYLTLNTESPRIYTKLRAQQCRFWTLFFPKVLEMTGNIDEAEQEWKAGFHRWNNYMMAWKNHFNDYTSKKERCAGF

SEQ ID NO: 5 - protein, 602 aa, Cat
MQSKGTIISIQFLLRFLLLWVLIGKSHTEEDIIITTKNGKVRGMNLPVLDGTVTAFLGIPYAQPPLGRLRFKKPQFLTKWSDIWNATKHANSCYQNADQSFPGFPGSEMWNPNTDLSEDCLYLNVWSPTPKPKNATVMIWIYGGGFQTGTSSLPVYDGKFLARVERVIVVSMNYRVGALGFLALPGNPEIPGNMGLFDQQLALQWVQKNIAAFGGNPKSVTLFGESAGAGSVSLHLLSPRSQPLFTRAILQSGSSNAPWAVMSLDEAKNRTLTLAKFIGCSKENDTEIIKCLRNKDPQEILLNELLVVPSDTLLSVNFGPVVDGDFLTDMPDTLLQLGQFKKTQILVGVNKDEGTAFLVYGAPGFSKDNDSIITRKEFQEGLKIYFPGVSEFGREAILFYYVDLLDDQRAEKYREALDDVLGDYNIICPALEFTTKFSELGNNAFFYYFEHRSSQLPWPEWMGVMHGYEIEFVFGLPLERRVNYTRAEEILSRSIMNYWANFAKYGNPNGTQNNSTRWPAFRSTDQKYLTLNAESPKVYTKLRAQQCRFWTLFFPKVLEMTGNIDEAEREWRAGFYRWNNYMMDWKNQFNDYTSKKESCAGL

SEQ ID NO: 6 - protein, 602 aa, Horse
MQSWGTIICIRILLRFLLLWVLIGNSHTEEDIIITTKNGKVRGMNLPVLGGTVTAFLGIPYAQPPLGRLRFKKPQSLTKWSNIWNATKYANSCYQNTDQSFPGFLGSEMWNPNTELSEDCLYLNVWIPAPKPKNATVMIWIYGGGFQTGTSSLPVYDGKFLARVERVIVVSMNYRVGALGFLALSENPEAPGNMGLFDQQLALQWVQKNIAAFGGNPRSVTLFGESAGAASVSLHLLSPRSQPLFTRAILQSGSSNAPWAVTSLYEARNRTLTLAKRMGCSRDNETEMIKCLRDKDPQEILLNEVFVVPYDTLLSVNFGPTVDGDFLTDMPDTLLQLGQFKRTQILVGVNKDEGTAFLVYGAPGFSKDNNSIITRKEFQEGLKIFFPRVSEFGRESILFHYMDWLDDQRAENYREALDDVVGDYNIICPALEFTKKFSELGNDAFFYYFEHRSTKLPWPEWMGVMHGYEIEFVFGLPLERRVNYTKAEEILSRSIMKRWANFAKYGNPNGTQSNSTRWPVFKSTEQKYLTLNTESPKVYTKLRAQQCRFWTLFFPKVLELTGNIDEAEREWKAGFHRWNNYMMDWKNQFNDYTSKKESCSDF

SEQ ID NO: 7 - protein, 603 aa, Mouse
MQTQHTKVTQTHFLLWILLLCMPFGKSHTEEDFIITTKTGRVRGLSMPVLGGTVTAFLGIPYAQPPLGSLRFKKPQPLNKWPDIHNATQYANSCYQNIDQAFPGFQGSEMWNPNTNLSEDCLYLNVWIRVPKPKNATVMVWIYGGGFQTGTSSLPVYDGKFLARVERVIVVSMNYRVGALGFLAFPGNPDAPGNMGLFDQQLALQWVQRNIAAFGGNPKSITIFGESAGAASVSLHLLCPQSYPLFTRAILESGSSNAPWAVKHPEEARNRTLTLAKFTGCSKENEMEMIKCLRSKDPQEILRNERFVLPSDSILSINFGPTVDGDFLTDMPHTLLQLGKVKKAQILVGVNKDEGTAFLVYGAPGFSKDNDSLITRKEFQEGLNMYFPGVSRLGKEAVLFYYVDWLGEQSPEVYRDALDDVIGDYNIICPALEFTKKFAELENNAFFYFFEHRSSKLPWPEWMGVMHGYEIEFVFGLPLGRRVNYTRAEEIFSRSIMKTWANFAKYGHPNGTQGNSTMWPVFTSTEQKYLTLNTEKSKIYSKLRAPQCQFWRLFFPKVLEMTGDIDETEQEWKAGFHRWSNYMMDWQNQFNDYTSKKESCTAL

SEQ ID NO: 8 - DNA, 1964 bp, Macaca mulatta
aacagttgattgctacattcagtaacactgaatgtcagtgcagtccaatttacaggctggagcagcagctgcatcctgcatttctctgaagtattgcatgattttcactccttgaaaaagtttaccacctttgttgcagagaatcggaaatcaatatgcatagcaaagtcacaatcatatgcatcagattactcttttggtttcttttgctctgcatgcttattggaaagtcacatactgaagatgacatcgtaattgcaacaaagaatggaaaagtcagagggatgaacttaacagttcttggtggcacggtaacagcctttcttggaattccctatgcacagccacctcttggtagacttcgattcaaaaagccacagtctctgaccaagtggtctgatatttggaatgccacaaaatatgcaaattcttgctatcagaacatagatcaaagttttccaggcttccatggatcagagatgtggaacccaaacactgacctcagtgaagactgtttatatctaaatgtatggattccggcacctaaaccaaaaaatgctactgtaatgatatggatttatggtggtggttttcagactggaacatcatctttacatgtttatgatggcaagtttctggctcgagttgaaagagttattgtagtgtcaatgaactatagggtgggtgcccttggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccatccattgttcaccagagccattctacaaagtggatcctctaatgctccttgggcagtaacatctctttatgaagctaggaacagaacattgaccttggctaaattgactggttgctctagagataatgagactgaaatagtcaagtgccttagaaataaagatccccacgaaattcttctgaatgaagcatttgttgtcccctatgggactctcttgtcagtaaacttcggtccaaccatggatggtgattttctcactgaaatgccagacatattacttgaacttggacaatttaaaaaaacccagatattggtgggtgttaataaagatgaagggacagcttttttagtctatggtgctcctggcttcagcaaagataacgatagtatcataactagaaacgaatttcaggaaggtttaaaaatattttttccaggcgtgagtgagtttggaaaggaatccatcctttttcattacacagactgggtagatgatcagagacctgaaaactaccgtgaggcgttggatgatgttgttggggattataatatcatatgccctgccttggagtttaccaagaagttctcagaatggggaaataatgcctttttctactattttgaacaccgatcctccaaacttccgtggccagaatggatgggagtgatgcatggctatgaaattgaattcgtctttggtttacctctggaaagaagagttaattacacaaaagctgaggaaattttgagtagatccatagtgaaacggtgggcaaattttgcaaaatatgggaatccaaatgggactcataataatagcacaaaatggcctgtcttcaaaagcactgaacaaaaatatctaaccttgaatacagagtcatcaagaatattgactaaactacgtgctcagcaatgccgattctggacatcattttttccaaaagtcttggaaatgacaggaaatattgatgaagcagaatgggagtggaaagcaggattccatcgctggagcaattacatgatggactggaaaaatcaatttaacgattacactagcaagaaagaaagttgtgtgggtctctaa

SEQ ID NO: 9 - DNA, 538 bp, Macaca mulatta (partial RhBchE, M62777)
aacatagatcaaagttttccaggcttccatggatcagagatgtggaacccaaacactgacctcagtgaagactgtttatatctaaatgtatggattccggcacctaaaccaaaaaatgctactgtattgatatggatttatggtggtggttttcagactggaacatcatctttacatgtttatgatggcaagtttctggctcgagttgaaagagttattgtagtgtcaatgaactatagggtgggtgcccttggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctagcaagaaagaaagttgtgtgggtctctaattaatagatttaccctttatagaacatttattttcctttagatcaaggcaaaaatatcaggagctttcttacacacctactaa

SEQ ID NO: 10 - DNA, 2196 bp, Homo sapiens (HuBchE (NCBI NM 000055)
agtaacagttgattgttacattcagtaacactgaatgtcagtgcagtccaatttacaggctggagcagcagctgcatcctgcatttccccgaagtattacatgattttcactccttgcaaactttgccatctttgttgcagagaatcggaaatcaatatgcatagcaaagtcacaatcatatgcatcagatttctcttttggtttcttttgctctgcatgcttattgggaagtcacatactgaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggatcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctctaattaatagatttaccctttatagaacatattttcctttagatcaaggcaaaaatatcaggagcttttttacacacctactaaaaaagttattatgtagctgaaacaaaaatgccagaaggataatattgattcctcacatctttaacttagtattttacctagcatttcaaaacccaaatggctagaacgtgtttaattaaatttcacaatataaagttctacagttaatt

SEQ ID NO: 11 - protein, 11 aa, Homo sapiens (partial sequence of the serine active site)
TLFGESAGAAS

SEQ ID NO: 12 - protein, 10 aa, Artificial Sequence
  the peptide used to develop OP-conjugate- selective anti-sera
LFGESAGAAC

SEQ ID NO: 13 - protein, 18 aa, Homo sapiens (Partial sequence of human albumin)
YKFQNALLVRYTKKVPQV

SEQ ID NO: 14 - protein, 18 aa, Rat (Partial sequence of rat albumin)
YGFQNAILVRYTQKAPQV

SEQ ID NO: 15 - protein, 18 aa, Mouse (Partial sequence of mouse albumin)
YGFQNAILVRYTQKAPQV

(19) United States
(12) Patent Application Publication
(10) Pub. No.: US 2007/0286846 A1
(43) Pub. Date: Dec. 13, 2007

(54) Novel methods for ester detoxication

(76) Inventors: John Cashman, San Diego, CA (US); Jun Zhang, San Diego, CA (US)

Correspondence Address: JONES DAY, 222 EAST 41ST ST, NEW YORK, NY, 10017, US

(21) Appl. No.: 11/810,257

(22) Filed: Jun. 4, 2007

Related U.S. Application Data
(60) Provisional application No. 60/811,370, filed on Jun. 7, 2006.

(51) Int. Cl. A61K 48/00; A61K 38/46; A62D 3/02; C12Q 1/34; C40B 50/06; C40B 40/08

(52) U.S. Cl. 424/93.2; 424/94.6; 435/262.5; 435/18; 506/4; 506/26

(57) ABSTRACT

This invention relates to a method for detoxication of inorganic or organic esters including OP nerve agents, cocaine, and respective analogs. More specifically, this invention pertains to the treatment of potentially neurotoxic esters or other ester groups by elaborating a more effective hydrolytic enzyme for therapeutic application. The structures of the synthesized OP analogs are provided. This invention also provides a diagnostic method and an Array Biosensor for detecting OP agents in biological and environmental samples.

### CROSS-REFERENCES TO RELATED APPLICATIONS

This application claims benefit to U.S. Provisional Application Ser. No. 60/811,370, filed on Jun. 7, 2006, which is incorporated herein by reference in its entirety.

### FIELD OF INVENTION

Provided herein are an isolated DNA molecule of monkey butyrylcholinesterase (RhBchE); an isolated DNA molecule of human butyrylcholinesterase (HuBchE) mutants; a mutation library featuring HuBchE and RhBchE; an expression vector containing DNA molecules of RhBchE and modified HuBchE; a high-level adenovirus (AD)-based expression system of Butyrylcholinesterase (BchE); organophosphate (OP) model compounds that mimic the structure of OP nerve agents; and use of OP model compounds to obtain antibodies for an array biosensor. The invention provides a general method for detoxication of inorganic or organic esters including organophosphate nerve agents, and a diagnostic method for detecting OP agents in biological samples and environmental samples.

### BACKGROUND OF THE INVENTION

Drug abuse is one of the major public health problems. More than 3 million Americans are heavy users and a like number of Americans are light abusers of cocaine. About 100,000 emergency room visits annually are cocaine-related. No effective treatment is available for the common complications of cocaine overdose on the cardiovascular and central nervous systems that produce cardiovascular distress and generalized seizures. Cocaine is an example of an organic ester that could be detoxicated in a therapeutic fashion by an esterase evolved utilizing the molecular evolution approach.

An example of inorganic ester that could be detoxicated in a therapeutic fashion are organophosphate (OP) nerve agents or pesticides. The biological threat from nerve agents exposure has also become of great concern as these compounds are able to block numerous vital enzymes. In particular, serine esterases and proteases are rapidly and irreversibly inhibited by OP nerve agents.

BchE is a soluble serum glycoprotein enzyme in the family of serine esterases. The physiological function of the enzyme is not clear. However, it has been known for decades that BchE scavenges low doses of OP and carbamate pesticides and protects people from the toxic effects of these poisons. BchE also serves as a marker for early detection of OP poisoning, because the exposure to OPs decreases the activity of the serum enzyme. BchE is also the primary enzyme for the metabolism and detoxication of cocaine and other esters in humans. BchE metabolizes cocaine to pharmacologically inactive compounds such as ecgonine methyl ester and benzoic acid. The enzyme poses great potential for use in cocaine detoxication and OP poisoning treatment.

Theoretically, BchE represents an ideal enzyme for enzyme supplementation therapy: it doesn't require cofactors, it is soluble and highly functional at the pH of the plasma; and the products of hydrolysis are non-toxic. Purified BchE is stable for years and has a relatively long half-life after exogenous administration. Pegylation of HuBchE markedly improves its stability and half-life. Most importantly, purified HuBchE has been used safely for decades in Europe for treatment of humans with succinylcholine-induced apnea and OP poisoning. Regardless of its theoretical potential, HuBchE is not a very efficient enzyme toward either OPs or cocaine hydrolysis. The irreversible binding of the enzyme for OPs limits the ability of the enzyme to work only as a scavenger and not as a catalyst. The enzyme hydrolyzes natural (−)-cocaine 2000-times slower than (+)-cocaine. Rationally designed mutants generated by protein engineering showed that the enzyme can be converted to an OP hydrolase (G117H and G117H/E197Q mutants described by Millard et al., 1995; Millard et al., 1998) as well as to cocaine hydrolase (A328W/Y332M/S287G/F227A mutant described by Pancook et al., 2003, Gao et al, 2005). While mutation studies suggest that improving hydrolysis activity of the enzyme is feasible, the mutants reported to date have low binding affinity and slow turnover and could be markedly improved. The catalytic flexibility of HuBchE offers great room for improvement of its OP and cocaine catalytic efficiency. However, combinations of HuBchE mutations at multiple residues, close to and/or far from the active site, are likely needed to significantly improve OP and/or cocaine hydrolysis activity. It is not easy to predict such combinations of mutations by molecular structure analysis due to the complicated multistep catalytic events associated with OP-enzyme interaction and serious stereo hindrance for (−)-cocaine hydrolysis. Application of molecular evolution technology to BchE will yield promising results for improving the catalytic activity for OP nerve agents and cocaine.

Current treatment of acute OP nerve agent poisoning generally includes a combined administration of a cholinesterase reactivator (oxime), a muscarinic receptor antagonist (atropine), and an anticonvulsant (diazepam). These treatments only act in a competitive fashion and are not adequate since they do not prevent neuronal brain damage and incapacitation. Chemical prophylactic treatments are introduced for use by military forces, including pyridostigmine bromide alone, pyridostigmine with antichoinergics, and HI-6 for transdermal administration (Bajgar, 2004).

Because the treatments for cocaine toxicity and OP poisoning are aimed to treat the symptoms rather than the source of the problem (i.e., the nerve agent and cocaine), an attractive alternative treatment approach is to apply a catalyst or scavenger to directly remove a nerve agent or cocaine before damage occurs. Fundamental to therapeutic treatment is the requirement to diagnose the amount and type of a nerve agent or OP that is present. A highly sensitive and inexpensive method to detect biomarkers of OP nerve agent exposure is essential in conjunction with medical protection against chemical warfare and other OP threats. Because available methods to detect biomarkers of nerve agents in animals before damage occurs are expensive and cumbersome, the challenge is to develop a fieldable and robust system. The present invention solves this problem by providing selective methods to detect biomarkers of OP nerve agents that can be readily and inexpensively deployed using a portable automated multianalyte array biosensor.

An array format offers a number of advantages, such as the potential to analyze a sample for a large number of targets simultaneously. Furthermore, inclusion of positive and negative controls on each sensing surface is more reliable than such controls located on parallel but separate sensing surfaces. DNA array technology has led this effort in terms of laboratory devices and two notable systems employing optical waveguides include the systems marketed by Zeptosens (Pawlak et al., 2002) and Illumina (Epstein and Walt, 2003). These systems accommodate thousands of capture molecules and are highly sensitive. However, they are designed for use by highly trained laboratory personnel and have not been automated or adapted for on-site applications. The Array Biosensor described in this invention combines optical waveguide technology and the capacity to test multiple samples simultaneously for multiple targets with portability and automation.

The biosensor is based on a planar waveguide with sufficient surface area to accommodate many small (mm²) sensing regions. The waveguide, a modified microscope slide, is illuminated using a 635 nm diode laser and a line generator, with the light launched into the proximal end. The first two-thirds of the slide provides a mode-mixing region so that the light is relatively uniform in the 2.4 cm² sensing region near the distal end (Feldstein et al., 1999). Under normal conditions, total internal reflection is achieved and an evanescent field is produced in the sensing region. The evanescent light excites fluorophores bound in the sensing region, and the emitted fluorescence is measured at 90° using a Peltier-cooled CCD camera (Wadkins et al., 1997; Golden et al., 2003). The location of the fluorescence within the array on the waveguide surface reveals the identity of the target detected. This system is commercially available.

In order to capture the target from the samples, antibodies or other molecules capable of binding to the target are immobilized on the waveguide surface in arrays of spots (Rowe et al., 1999; Delehanty et al., 2002). Both positive and negative controls can be included in the arrays to prevent false-positive or false-negative responses (Ligler et al., 2003). Furthermore, the use of multiple channels in combination with the arrays of sensor spots enables the analysis of multiple samples simultaneously. Assays can be formatted to detect either large molecules and microorganisms (sandwich assays) or small molecules (competitive assays, displacement assays) (Sapsford et al., 2002). The use of near-infrared fluorescence prevents interference from sample components, which may autofluoresce at shorter wavelengths, making separation of the target from complex samples unnecessary prior to analysis (Sapsford et al., 2001; Taitt et al., 2004). In contrast to mass-sensitive sensors, such as the surface plasmon resonance (SPR), resonant mirror, or interferometric systems (Homola et al., 2002; Kinning and Edwards, 2002; Campbell and McCloskey, 2002, Barzen et al., 2002), the fluorescence-based Array Biosensor requires a fluorophore-labeled molecule for signal generation. This makes the assay relatively immune to interference from nonspecific adsorption by sample components (Ligler et al., 2003; Rowe et al., 1999; Sapsford et al., 2001; Taitt et al., 2004).

The capacity of the Array Biosensor to detect biomarkers in complex samples with little or no sample preparation has already been shown using a non-automated prototype. The invention herein applies the automated system to determine how effectively the system can detect nerve agent-related or other biomarkers both in the laboratory and the field setting.

### SUMMARY OF THE INVENTION

In one aspect, the invention provides an isolated DNA molecule of monkey rhesus butyrylcholinesterase (RhBchE) (Macaca mulatta).

In another aspect, the invention provides an isolated DNA molecule of human butyrylcholinesterase (HuBchE) and mutants thereof.

In another aspect, the invention provides a mutation library featuring HuBchE and RhBchE and a method for making a mutation library of BchE in a vector.

In yet another aspect, the invention provides a method of infecting a cell with a vector by packaging of a mutation library of BchE in a vector particle

In another aspect, the invention provides an expression vector containing DNA molecules of RhBchE and modified HuBchE; and a high-level adenovirus (AD)-based expression system of BchE.

In another aspect, the invention provides racemic as well as enantiomerically pure OP model compounds that mimic the structure of OP nerve gases such as VX, tabun, GF, soman, and sarin.

In another aspect, the invention provides means for use of the OP model compounds in a cell-based functional screening assay to identify OP resistant and/or catalytic BchE variants expressed from the BchE mutation library.

In yet another aspect, the invention provides means for use of OP model compounds to obtain antibodies for the array biosensor.

In another aspect, the invention provides a diagnostic method for detecting OP agents in biological samples.

In further aspect, the invention provides a method for screening an OP model compound for BchE activity by incubating the BchE with the compound and further detecting the inhibition of BchE as indication of the activity of the compound.

In yet another aspect, the invention provides a method for detecting BchE expression in culture medium of infected cells.

In another aspect, the invention provides a method for detecting BchE by prokaryotic or eukaryotic cells expressing BchE in the presence of an OP compound or a nerve agent.

In another aspect, the invention provides an array-based methodology to selectively detect nerve agents and other OPs.

### BRIEF DESCRIPTION OF THE FIGURES

FIG. 1A shows RhBchE transfection. CHO cells were seeded overnight in 6-well plates. Plasmids containing background vector (N.C.), pRC-CMV-HuBchE (hBchE), pGS-RhBchE (GS1 and GS2), and pRC-CMV-RhBchE (RC1 and RC2) were transfected into to the cells with Lipofectamine as described by the manufacturer. The medium was collected at 24 hr and 48 hr post-transfection, and used for BchE activity assay using Ellman assay with 1 mM BchI as substrate. FIG. 1B shows the results of western blot analysis of RhBchE. The BchE unit is defined as amount of enzyme that hydrolyzes 1 μmol substrate per min. Samples from 48 hr post-transfection were analyzed with western blot analysis using a polyclonal antibody developed against HuBchE (B).

FIG. 2A. Purification of RhBchE. SDS-PAGE of purified RhBchE was detected with commassie blue. FIG. 2B. SDS-PAGE of purified RhBchE was detected with anti-HuBchE antibody.

FIG. 3A. Inhibition of RhBchE by the indicated concentrations of echothiophate (ETP). FIG. 3B. Inhibition of HuBchE by the indicated concentrations of echothiophate. Data were fit to a first-order process using Prism. The slope yields kₐₚₚ (min⁻¹) for a given ETP concentration. Re-plot of the kₐₚₚ (min⁻¹) over the ETP concentration used for initial inhibition incubation for ETP yielded the data reported in Table 5.

FIG. 4A. Inhibition of HuBchE by the specific concentrations of compound 1. FIG. 4B. Inhibition of HuBchE by the specific concentrations of compound 2. FIG. 4C. Inhibition of HuBchE by the specific concentrations of compound 3. FIG. 4D. Inhibition of HuBchE by the specific concentrations of ETP. Data were fit to a first-order process using Prism. The slope yields kₐₚₚ (min⁻¹) for a given OP concentration. FIG. 4E. Plot of the kₐₚₚ (min⁻¹) over the OP concentration used for initial inhibition incubation for compound 1. The re-plot gave a linear fit. The slope yields the k_{inact}/Kᵢ=4.1×10³ M⁻¹min⁻¹. Double reciprocal plot of the data yielded the data reported in Table 7.

FIG. 5. Diagram of eight randomly selected representative clones of gene shuffled recombinant BchEs.

FIG. 6A shows Butyrylthiocholine iodide (BchI) hydrolysis activity. COS cells were pre-seeded in 6-well plates overnight. The cells were then infected with mock infection (Control) or 1×10⁸ pfu and 4×10⁸ pfu HuBchE-AD per well specifically at time 0. BchI hydrolysis activity was examined for culture medium at the indicated time, post infection. A BchE unit was defined as enzyme needed to hydrolyze 1 μM/min BchI substrate. FIG. 6B shows the results of Western blot analysis of adenovirus (AD) expression vector meadiated BchE expression. Culture medium samples collected at indicated time post infection were analyzed on SDS-PAGE. 0, 1, and 2 represent cells infected by mock infection (Control) or 1×10⁸ pfu and 4×10⁸ pfu HuBchE-AD, respectively. HuBchE in the medium was detected with western blot analysis.

FIG. 7 shows the BchE activity of 293A cells transfected with PacI linearized indicated recombinant AD. BchE activity in the culture medium collected at indicated time post-transfection was analyzed with Ellman method.

FIG. 8A shows cell based HuBchE expression mediated BchI hydrolysis in the solid-phase assay using different solid matrix between 0-3 hour. FIG. 8B shows the BchI hydrolysis in the same solid-phase assay as in 8A after 3.5-7.3 hr incubation. CHO-K1 cells stably expressing WT HuBchE were seeded in 24 well plates overnight. The cells were treated as described in the text. Absorbance at 405 nm in each well was recorded by a plate reader over time. The legend indicates the solid-phase material used for cell coating (1st layer) and substrate application (2nd layer).

FIG. 9A shows the localization of the HuBchE expressing cells. The plates seeded with CHO-K1 cells stably expressed WT HuBchE were developed with an Ellman mixture with BchI substrate as described in text. FIG. 9B shows an inverted image of plate A.

FIG. 10 shows an illustration of specific plugs isolated during virus isolation from identified yellow spots. 293A cells in 6-well plate were infected with serial diluted HuBchE and stained for BchE activities as described. Indicated positive plugs, negative plugs, sample plugs, and background plugs were cored out from the plate and transferred to medium for virus extraction.

### DETAILED DESCRIPTION OF THE INVENTION

The invention provides an isolated DNA molecule of monkey rhesus butyrylcholinesterase (RhBchE) (Macaca mulatta). The RhBchE was cloned from RNA using the RACE kit and RT-PCR. The full length RhBchE was cloned into the HindIII/ApaI sites of pRC-CMV. Obtained plasmids were transfected into CHO cells and the expression of active BchE enzyme was monitored from the assay medium for BchI hydrolysis. Also provided is an isolated DNA molecule of HuBchE and mutants thereof.

The invention provides a mutation library featuring HuBchE and RhBchE, a method for making a mutation library of BchE in a vector, and a method of infecting a cell with a vector by packaging of a mutation library of BchE in a vector particle. Further provided is an expression vector containing DNA molecules of RhBchE and modified HuBchE and a high-level adenovirus (AD)-based expression system of butyrylcholinesterase. In the methods of the invention, the vector may be any vector suitable for this purpose. In a preferred embodiment, the vector is a pENTRA vector or an adenovirus vector. The cell may be any cell, preferably a mammalian cell.

To create a BchE library, a site-saturation mutagenesis technology was used. The mutagenesis can be performed at any chosen specific position, (e.g. position G117 and E197 for HuBchE library). Site-saturation mutagenesis is carried out to incorporate NNK randomly mutagenized codons (N=A, T, C, or G; and K=G or T) to replace specific positions of HuBchE using a two-step PCR. The PCR product then can be cloned into the pENTRA 1 vector through KpnI/XhoI sites. Plasmid DNA from pooled pENTRA-HuBchE clones can be used for recombination with an pAD/CMV/V5/DEST vector. Plasmids of the pAD-HuBchE mutation library pool can be digested with PacI enzyme and then transfected into 293A cells using Lipofectamine 2000 for AD packing. A recombinant AD-HuBchE viral library can be collected from cell supernatant. The library is then screened with both a primary high-throughput solid phase functional screen and a secondary liquid based activity screen (Example 4).

Use of the RhBchE or the mutants of the HuBchE having one or all of the RhBchE amino acid changes listed in Table 1 by site-directed mutagenesis or other means should provide an enzyme with greater hydrolytic activity. These residues could be responsible individually or in combination for the higher substrate binding affinity and velocity seen in the purified native enzyme. The different residues could alter the folding of the protein and/or the post-translational modification of the protein (i.e., N-glycosylation, phosphorylation), and therefore change the recognition and entry of cocaine or other esters to the binding site of the enzyme, the actual hydrolysis of cocaine, or the release of the hydrolysis products. Many of the different residues present in the C-terminal part of the protein could possibly alter the dimerization or tetramerization of the proteins and therefore alter the stability of the enzyme. Mutations of the HuBchE based on information obtained from RhBchE may therefore produce an enzyme with high hydrolytic activity. It is possible that the variation in primary sequence by the RhBchE may provide some selective advantage to monkey. Molecular evolution of HuBchE utilizing amino acid variation present in RhBchE, as well as randomly or selectively introduced mutations, could provide a highly selective and potent catalyst to remove potentially toxic esters (or their chemical homologues) from the blood stream. Provided herein, the functional screening system has been fully validated and ensures the successful molecular evolution of such a product.

Further provided is a method for detecting BchE expression in culture medium of infected cells. The cell culture may be any cell culture suitable for the methods of the invention. In a preferred embodiment, the cell culture is a mammalian cell culture. The expression system for BchE may be any expression system suitable for such purpose. In a preferred embodiment, the expression system is a high level adenovirus(AD)-based expression system capable of incorporating mutation library expression and adapting to high throughput format functional screening. AD can infect a broad range of mammalian cells and permit expression of various proteins in different dividing and non-dividing cell lines (Example 10).

The invention provides OP model compounds that mimic the structure of OP nerve agents such as VX, GF, tabun, soman, and sarin. In the methods of the invention, the compounds are used in a concentration from 0.01 to 20 mM, preferrably in a concentration from 0.1 to 10 mM. In a specific embodiment, the concentration of a compound is 0.5 mM. The synthesis of the compounds is described in detail in Examples 12 & 13.

The invention provides a method for screening an OP model compound for BchE activity by incubating the BchE with a compound and further detecting the inhibition of BchE as indication of the activity of the compound. The cells expressing BchE in the presence of an OP compound or a nerve agent may be prokaryotic or eukaryotic cells.

The invention provides a diagnostic method for detecting OP agents in biological samples. As the acute toxic effects of OP compounds correlate well with their ability to inhibit AChE by reaction with an essential serine hydroxyl to form a relatively stable phosphoserine ester bond, the OP-ChE conjugates can serve as exquisitely sensitive selective markers of OP exposure. Likewise, phosphorylated albumin (i.e., Tyr 411) can serve as a sensitive marker of OP or pesticide exposure. By using selective antibodies to specifically recognize individual OP-ChE or OP-albumin conjugates based on the precise modification imposed by the specific OP compound, the tool of enormous diagnostic value is obtained in ascertaining the relative toxic potential of an OP during or after an exposure.

The invention further provides means for use of OP model compounds to obtain antibodies for the array biosensor by using an array-based methodology to selectively detect nerve agents and other OPs. This is a three-pronged approach. First, synthesized chemical reagents are used for procuring the antibodies; second, the antibodies are obtained and incorporated into the Biosensor and tested in different assay formats to determine the optimal configuration for sensitive detection and optimized for both sensitivity and rapid detection; and third, following demonstration of such selectivity in buffer, spiked physiological fluids and finally blood, blood components, or brain tissue of an animal treated at physiologically relevant concentration is tested again for sensitivity and selectivity. The final product is a fieldable Array Biosensor of use in detecting OPs in environmental samples or biological samples taken from animals exposed to low doses of nerve agents or other OPs (Example 14).

### METHODS OF USE THE INVENTION

The invention provides a clinically tested recombinant HuBchE (and/or a more active catalytic variant forms of this enzyme) as an efficient biological scavenger (and/or enzymatic catalyst) useful to remove nerve agents, cocaine, pesticides, other drugs of abuse in vivo before damage occurs. The invention represents a promising approach to protect military personnel, first responders and civilians from the threat of OP exposure and provides an emergency treatment for cocaine or other agent overdose. This product can also be used to protect people potentially exposed to pesticides. In addition to human clinical use, the product can also be used as a detoxication device, such as nerve agent detoxication sponge, which absorbs and detoxifies nerve agents on skin or other surfaces; a detection device, such as testing strips to provide fast and sensitive detection of nerve agents; and a decontamination reagent for destruction and disposal of nerve agents.

Besides the final product, the OP analogs and the OP hydrolysis assays provided herein can be readily applied to other enzymes such as paraoxonase, carboxylase, and catalytic antibodies; the diversified library can be used for screening for other commercially viable biotherapeutics (e.g., cocaine hydrolysis) and industrial enzymes.

The invention is further described by the following non-limiting examples.

### EXAMPLE 1

### Cloning, Expression and Purification of RhBchE

Construction of RhBchE full length expression vector. The 5′ sequence of the RhBchE was cloned directly from rhesus monkey (Macaca mulatta) RNA using the RACE kit following the manufacture's procedure. Specifically, total RNA was prepared from a sample containing livers of three rhesus monkeys. After dephosphorylation of short mRNA, removing the cap structure of full length mRNA, an RNA RACE oligo was ligated onto the full length mRNA. Then through RT-PCR and cloning, the 5′ sequence of the RhBchE was obtained. The 3′ RhBchE sequence was confirmed through a recent input sequence (NCBI BV211040) and disclosed in the provisional application Ser. No. 60/811,370, filed Jun. 7, 2006, incorporated by reference in its entirety. The primer was designed based on the obtained sequence. The full length RhBchE, including the RhBchE signal peptide region and the mature BchE, was amplified through PCR and cloned into the HindIII/ApaI sites of pRC-CMV as well as the pGS vector. The pGS vector was essentially the same as the pRC-CMV vector, except that the selection marker of G418 in pRC-CMV was replaced with rat glutamine synthetase. The sequence of the cloned plasmid was confirmed.

Expression of functional recombinant RhBchE. Plasmids were transfected into CHO cells and expression of active BchE enzyme was monitored from the assay medium for BchI hydrolysis using the Ellman reaction. The expression of the BchE protein was also confirmed by western blot analysis with a rabbit anti-HuBchE polyclonal antibody.

Stable cell line selection. To prepare stable cell lines that provide high level expression of recombinant RhBchE, CHO cells were transfected with the pGS-RhBchE vector. At 24 hr post-transfection, the cells were trypsinized and diluted 10-fold and 20-fold before plating on 15 cm dishes. Selection medium used is serum free Ultraculture medium without L-glutamine containing 25 μM methionine sulfoximine (MSX). MSX is a specific inhibitor of internal expressed glutamine synthetase. Therefore only cells carrying the pGS-RhBchE vector can replicate under this selective condition. The transfected cells were kept in the selective medium for 2 weeks to allow colony formation. Twenty four single colonies separated from the others were randomly selected. Culture medium was removed from the culture plates. Small filter paper soaked with trypsin was applied to selected colonies and incubated at room temperature for 2 minutes. The filter paper was then transferred to individual wells in 24-well plates provided with the selective medium indicated above. Cells were allowed to grow to confluence. The medium of each well was assayed for BchE activity to identify stable cell lines that provided high level of expression.

Preparation of affinity resin. For purification of RhBchE, a procainamide conjugated sepharose 4B column was made following the procedure described (Grunwald et al, 1997) with slight modification. CNBr-activated sepharose 4B fast flow) was washed thoroughly with 1 mM HCl (15 times wash with 1 volume) to remove carry-over sugar. The resin was then resuspended in coupling buffer 0.2 M NaCO3 pH 9.0 containing 0.4 M NaCl to adjuct to pH 9. After removing the coupling buffer, 0.5 volume of coupling buffer containing 0.1 M γ-aminocapronic acid was added and allowed the reaction to rotate at 4° C. overnight (˜20 hr). The medium was washed with H₂O 5 times, and the resin was resuspended in 0.1 M 1-(3-dimethylaminopropyl)-3-ethylcarbodiimide hydrochloride and adjusted to pH 4.5 with HCl, and the liquid was removed. The resin was then resuspended in 0.5 volume of 0.1 M 1-(3-dimethylaminopropyl)-3-ethylcarbodimide hydrochloride containing procainamide at a concentration of 100 mol/ml of resin with the addition of 1 M HCl to maintain the pH 4.5 for 2.5 hr. In the reaction, the pH is adjusted every 5 min and the pH ranged about pH 4.45 to 4.65. The reaction was allowed to complete at room temperature overnight (˜20 hr). The resin was then packed in a column and washed thoroughly with H₂O with monitoring at UV280 for complete removal of unreacted procainamide. All flow-through was collected to measure un-coupled procainamide.

Purification of recombinant RhBchE from culture medium. To prepare purified recombinant protein, stable cells expressing RhBchE were grown in T180 flasks that allowed three layers of cell growth. Medium from the flasks was collected after two weeks of accumulation of secreted RhBchE. The medium was incubated with procainamide-resin overnight at 4° C. with constant rotation. The resins were settled by 5 min centrifugation at 2000×g. The supernantant containing unbound materials was carefully removed and the resins were resuspended in 50 mM potassium phosphate pH 7.2, 1 mM EDTA (Buffer A) and packed into a column. After extensive washing with 0.2 M NaCl in buffer A, the RhBchE bound to the column was eluted with 0.05 to 0.5 M procainamide gradient solution in buffer A. Active fractions from the eluate were identified by the Ellman assay using BchI substrate. The active fractions were pooled together and concentrated with Centricon (Millipore) with Molecular weight cut off of 30 kD.

Purification of native BchE from M. mulatta serum. Serum from M. mulatta was passed through a procainamide-conjugated sepharose column. The column was washed thoroughly with 0.2 M NaCl in 20 mM potassium phosphate pH 7.0, 1 mM EDTA. The RhBchE bound to the column was eluted with 0.1 M procainamide. The activity of the enzyme fractions was determed by BchI hydrolysis. Active fractions were pooled together and purified further on Sepharose 4B gel filtration chromatography in 20 mM Tris pH 8.0, 1 mM EDTA. Based on BchI hydrolysis activity, active fractions were pooled together and loaded on a DEAE sepharose column. The column was thoroughly washed with 20 mM Tris pH 8.0, 1 mM EDTA and protein bound to the column were eluted by step gradients of 0.1 M, 0.15 M, 0.2 M, 0.25 M, and 0.3 M NaCl in 20 mM Tris pH 8.0, 1 mM EDTA. The active enzyme was present in the 0.2 M NaCl eluant. The protein concentration was determined using the BCA method. The protein in each fraction was analyzed by SDS-PAGE followed by commassie blue staining and western blot analysis.

### EXAMPLE 2

### Evaluation of Substrate Specificity and Inhibition Kinetics for RhBchE and HuBchE

Hydrolysis of BchI. Enzyme fractions were analyzed for BchI hydrolysis using the Ellman method. Briefly, 5 mM BchI were incubated with the serum in 50 mM potassium phosphate pH 7.4 at 25° C. in the presence of 10 mM 5,5′-dithiobis(2-nitrobenzoic acid) (DTNB). Hydrolysis of BchI was monitored continuously at 412 nm with a UV-Vis spectrophotometer. Activity was calculated from the molar extinction coefficient of 13,600 M⁻¹ cm⁻¹. For Kₘ determination, the assays contained 25, 33.3, 50, 100, and 200 μM of BchI, respectively, enzyme stock, and 50 mM potassium phosphate pH 7.2 buffer with 200 μM DTNB. The assay was carried out at 25° C. Kₘ values were determined by Lineweaver-Burk analysis, and k_{cat} values were determined using the functional enzyme concentrations determined from echothiophate (ETP) titration. The competitive inhibition constant, Kᵢ, of (+)-cocaine, (−)-cocaine and some of its metabolites was determined by measuring hydrolysis of BchI in the presence of (+)-cocaine, (−)-cocaine, (−)-norcocaine, at a range of concentrations.

Cocaine Hydrolysis Assay. Cocaine hydrolysis was characterized by quantifying specific ecgonine methylester (EME) production by mass spectrometry (MS). Highly purified RhBchE or HuBchE was incubated with cocaine (1, 2, 4, 10, and 40 μM final concentration in 10 mM potassium phosphate buffer pH 7.4) at 37° C. At intervals of 20, 40, and 60 minutes, aliquots were mixed with 6 N HCl to stop the reaction and stabilize the reaction products. Background reactions were performed simultaneously without added enzyme. The amount of EME produced at each time point was determined using flow injection of reaction aliquots directly into an Agilent MSD model MS, and ions of m/z 199.9-200.9 (EME) were quantified by selective ion monitoring. The amount of EME in the reaction mixtures was compared to a standard curve generated from EME solutions of known concentrations (125-1000 nM). The specific enzyme-catalyzed rate at each cocaine concentration was determined by subtracting the corresponding background rate. Kₘ and Vₘₐₓ values were determined by Lineweaver-Burk analysis, and k_{cat} was determined using the functional enzyme concentrations determined from echothiophate (ETP) titration.

Inhibition kinetics of RhBchE and HuBchE by OP compound. The kinetics of time-dependent inhibition of purified RhBchE and HuBchE by model OP compounds and ETP was studied in 50 mM potassium phosphate buffer pH 7.2 at 25° C. Inhibition of RhBchE and HuBchE was initiated by mixing highly purified HuBchE and RhBchE with various amounts of ETP. At defined times, the reaction mix containing 1 mM BchI and 0.2 mM DTNB was added to the enzyme-compound mix and hydrolysis of BchI was measured to determine residual BchE activity. Seven inhibitor concentrations were used for the assay with five time points for each inhibitor concentration.

### EXAMPLE 3

### Characterization of HuBchE Interaction with Novel OP Compounds

Inhibition of WT and G117H/E197Q HuBchE by OP analogues. WT or G117H/E197Q HuBchE were individually incubated with 0.5 mM of compounds 1, 2, 3 (or 4-13, see Example 12) or ETP at 4° C. for 48 hrs. Standard substrate BchI (1 mM) was then used to measure percent of remaining enzyme activity using the Ellman method after 100-fold dilution of the original enzyme-compound incubation mixture.

Inhibition rate constant determination for inhibition of HuBchE by model OP compounds. The kinetics for time-dependent inhibition of purified HuBchE by the model OP compounds was studied in 50 mM potassium phosphate buffer pH 7.2 at 25° C. Inhibition of HuBchE was initiated by mixing 15 nM of highly purified HuBchE with various amounts of compounds 1, 2, 3, (or 4-13, see Example 12) or ETP. At defined times, the reaction mixture containing 1 mM BchI and 0.2 mM DTNB was added to the enzyme-compound mixture and hydrolysis of BchI was measured to determine residual HuBchE activity. Seven inhibitor concentrations were used for the assay and five time points of enzyme activity were determined for each inhibitor concentration.

### EXAMPLE 4

### Human BchE Mutant Expression Library Construction

Construction of a mutation library of HuBchE. Site-saturation mutagenesis technology has been used to create a HuBchE library at position G117 and E197. Site-saturation mutagenesis was carried out to incorporate NNK randomly mutagenized codons (N=A, T, C, or G; and K=G or T) to replace G117 and E197 positions of huBuChE using a two-step PCR. The PCR product was cloned into the pENTRA 1 vector through KpnI/XhoI sites. Plasmid DNA from pooled pENTRA-huBchE clones were used for recombination with an pAD/CMV/V5/DEST vector. Plasmids of the pAD-huBchE mutation library pool were digested with PacI enzyme to expose the left and right viral ITRs and then transfected into 293A cells using Lipofectamine 2000 for AD packing. A recombinant AD-huBuChE viral library was collected from cell supernatant 5 days post transfection. The library was then screened with both a primary high-throughput solid phase functional screen and a secondary liquid based activity screen.

As an example, the functional screening platform was validated using the two position (G117 and E197) site-saturation mutagenesis-generated library above. The library recombinant viruses were used to infect 293A cells, and the cells were coated with 1% agarose in MEM 24 hr post infection, then 0.4 mM of compound 4-13 (i.e., compound 5, sarin analog, see Example 13) was added to the coated cells to interact with expressed recombinant HuBchE variants. At day 4 post infection, the coated cells were stained using BchI as substrates in the presence of DTNB. The plates were monitored for 3 hrs for appearance of yellow spots, which indicate the presence of OP-hydrolyzing enzyme. The yellow agarose spot(s) were cored out from the plate and incubated in serum-free medium in individual tubes. Aliquots from the recombinant viruses released in the medium were added to 96-well plate pre-seeded with 293A cells. The 96-well culture plate was incubated for 3 days post-infection. This time allowed the multiplication of virus and expression of the encoded huBuChE variants. On day 3, culture medium from the 96-well plate was assayed for OP inhibition resistance/hydrolysis activity. Based on the results of all the above assays, the samples showing increased OP hydrolysis activity were selected for gene identification. The huBchE gene encoded in the recombinant virus was PCR amplified with viral vector specific primers from the culture medium. The PCR products were sequenced and mutations in the huBchE gene were identified.

AD-mediated expression of HuBchE. A stock of recombinant AD containing a variant of HuBchE (A328Y/Y332A) with a titer of 2.45×10¹⁰ pfu/ml. This double mutant variant was designed for improved cocaine hydrolysis. Two different concentrations (1×10⁸ pfu and 4×10⁸ pfu, (i.e. 200 multiplicity of infection (moi) and 800 moi respectively) of virus were used to infect CHO cells and COS cells pre-seeded overnight onto a 6-well plate. BchE activity in the culture medium was monitored at different time points post infection using 1 mM BchI as substrate using standard Ellman reaction.

Preparation of AD expression vector for WT and G117H/E197Q HuBchE. A ViralPower AD Expression system from Invitrogen was used to clone the HuBchE enzyme. The system involved the cloning of the target gene in an entry vector. The target gene was transferred to the pAD/CMV/V5/DEST through in vitro recombination using the Gateway technology as described by the manufacturer. To clone the HuBchE gene into the pENTRA1 entry vector, which provides the recombination signal needed for the construction of the adenoviral vector, PCR-amplified WT and G117H/E197Q HuBchE from the original plasmid were made using Turbo Pfu to incorporate the restriction cloning sites KpnI and XhoI. The PCR product was cloned in the KpnI and XhoI sites of pENTRA1. The insert of selected clones was sequenced to verify that no mutations were introduced through the PCR step. The supercoiled plasmid was then incubated with the pAD/CMV/V5/DEST, the AD expression vector containing a CMV promoter for high level target protein expression and negative selection ccdB gene. After transformation, only recombined plasmids were able to grow on the selection plate. Plasmids were analyzed by restriction digestion and sequencing analysis to verify the correct insertion of HuBchE gene in pAD vector.

Generation of HuBchE-AD and validation of expression of recombinant WT and G117H/E197Q HuBchE with the AD expression system. To generate the recombinant WT- and G117H/E197Q-hBchE-AD, plasmids pAD-WT-hBchE and pAD-G117H/E197Q-hBchE were linearized with PacI enzyme digestion, and then transfected into 293A cells using Lipofectamine. The transfected cells were kept in serum-free medium for three days to examine the HuBchE activity, and then transferred to serum-containing medium to help maintain cell viability. Control vector pAD-lacZ was also used in parallel. The medium containing packaged recombinant virus was collected at day 12 post transfection, centrifuged to remove cell debris and stored in −80° C. To monitor recombinant BchE expression post infection, recombinant virus was added into the medium of cultured COS cells. BchI hydrolysis activity was determined for the culture medium at different time points post initial infection.

Construction of a mutation library of RhBchE and HuBchE chimeras. DNA shuffling technology was used to create BchE chimera libraries. Full length fragments of HuBchE and RhBchE were PCR amplified from the corresponding clones. The PCR fragments of HuBchE and RhBchE were gel purified and combined in a 1:1 ratio. The combined PCR fragments were subjected to limited DNase I digestion. Fragments of 100-200 bp were purified and PCR amplified without primer to assemble into longer fragments. The longer fragments were finally amplified with end primers to obtain the full length recombinant product. These shuffled DNA fragments were ligated into plasmid pCR2.1-TOPO.

### EXAMPLE 5

### Development and Validation of Functional Screening Assays

Establish a solid-phase cholinesterase activity assay. CHO-K1 cells stably expressing the HuBchE WT or G117H/E197Q HuBchE were seeded in culture plates. At 24 hr post seeding, the culture medium was removed and the cells were washed twice with Ultraculture serum-free medium. The cells were then overlaid with 1% agar or 1% agarose in colorless MEM. After the medium solidified, the cells were returned to the incubator overnight. The Ellman reaction mixture containing substrate (1 mM BchI or 1 mM ETP) and/or 0.1 mM DTNB were prepared in 1% agar (or 1% agarose) in colorless MEM. The reaction mixture was overlaid on cell culture plates prepared above and incubated at room temperature. The plates were visually monitored for appearance of yellow color and were also measured for OD405 absorption with a plate reader.

Localized detection of HuBchE activity. CHO-K1 cells stably expressing WT HuBchE were serial diluted and seeded onto 10 cm culture dishes at different densities (i.e., 4, 20, and 100 cells/plate). The cells were allowed to grow for 1 week to form small colonies containing ˜20 cells/colony. The cells were washed 2 times with serum free culture medium and then coated with 1% agarose in MEM. After overnight incubation, the plates were developed with an Ellman reagent mixture containing substrate (1 mM BchI and 0.1 mM DTNB) prepared in 1% agarose in colorless MEM.

Application of a solid-phase BchE activity assay to the detection of AD-BchE recombinant virus. Serial-diluted HuBchE-AD virus was used to infect 293A cells pre-seeded in 6-well plates. An agarose-MEM mixture was overlaid on cells at 1 hr post infection. The culture plates were returned to the incubator. Multiple duplicated plates were prepared, so on each additional day after infection one plate was taken out and overlaid with agarose-MEM mixture containing 1 mM BchI and 200 μM DTNB. Appearance of yellow color on plates was visually monitored. To isolate virus from the identified yellow spots, plugs were cored-out from the agarose culture plate. The isolated plugs were transferred to tubes containing 0.5 ml of cultured medium and incubated at 4° C. overnight. Medium incubated with plugs was then used to infect 293A cells pre-seeded in 24-well plate. BchE activity in the medium was assayed at 24 and 48 hr post-infection. To recover the BchE gene from the isolated recombinant virus, 1 μl medium incubated with plugs was used as a template and amplified with AD vector specific primers T7 and pAD-V5R. Sequence of the PCR product was determined.

### EXAMPLE 6

### Activity Analysis of Macaca ChE

To identify novel BchE with greater cocaine hydrolytic or OP activity, plasma from 3 monkeys species: Macaca mulatta, Macaca nemestrina and Macaca fascicularis were screened for BchI and cocaine hydrolytic activity. The study showed that while the BchI hydrolysis was very similar for all plasma, hydrolysis of (−)-cocaine was significantly different, and the M. mulatta serum possessed the greatest hydrolytic activity. Therefore, the BchE gene from M. mulatta was cloned to further characterize the enzyme.

Analysis of the cDNA Sequence of RhBchE. RT-PCR was used to clone the cDNA for RhBchE from M. mulatta liver tissue. The complete cDNA sequence of RhBchE (SEQ ID NO:1) and corresponding amino acid sequence (SEQ ID NO:2) are shown below.

| | M H S K V T I I C I R L L |
|---|---|
| | F W F L L L C M L I G K S |
| | H T E D |
| 1 | ATGCATAGCA AAGTCACAAT CATATGCATC AGATTACTCT |
| | TTTGGTTTCT TTTGCTCTGC ATGCTTATTG GAAAGTCACA |
| | TACTGAAGAT |
| | D I V I A T K N G K V R G |
| | M N L T V L G G T V T A F |
| | L G I P |
| 91 | GACATCGTAA TTGCAACAAA GAATGGAAAA GTCAGAGGGA |
| | TGAACTTAAC AGTTCTTGGT GGCACGGTAA CAGCCTTTCT |
| | TGGAATTCCC |
| | Y A Q P P L G R L R F K K |
| | P Q S L T K W S D I W N A |
| | T K Y A |
| 181 | TATGCACAGC CACCTCTTGG TAGACTTCGA TTCAAAAAGC |
| | CACAGTCTCT GACCAAGTGG TCTGATATTT GGAATGCCAC |
| | AAAATATGCA |
| | N S C Y Q N I D Q S F P G |
| | F H G S E M W N P N T D L |
| | S E D C |
| 271 | AATTCTTGCT ATCAGAACAT AGATCAAAGT TTTCCAGGCT |
| | TCCATGGATC AGAGATGTGG AACCCAAACA CTGACCTCAG |
| | TGAAGACTGT |
| | L Y L N V W I P A P K P K |
| | N A T V M I W I Y G G G F |
| | Q T G T |
| 361 | TTATATCTAA ATGTATGGAT TCCGGCACCT AAACCAAAAA |
| | ATGCTACTGT AATGATATGG ATTTATGGTG GTGGTTTTCA |
| | GACTGGAACA |
| | S S L H V Y D G K F L A R |
| | V E R V I V V S M N Y R V |
| | G A L G |
| 451 | TCATCTTTAC ATGTTTATGA TGGCAAGTTT CTGGCTCGAG |
| | TTGAAAGAGT TATTGTAGTG TCAATGAACT ATAGGGTGGG |
| | TGCCCTTGGA |
| | F L A L P G N P E A P G N |
| | M G L F D Q Q L A L Q W V |
| | Q K N I |
| 541 | TTCTTAGCTT TGCCAGGAAA TCCTGAGGCT CCAGGGAACA |
| | TGGGTTTATT TGATCAACAG TTGGCTCTTC AGTGGGTTCA |
| | AAAAAATATA |
| | A A F G G N P K S V T L F |
| | G E S A G A A S V S L H L |
| | L S P G |
| 631 | GCAGCCTTTG GTGGAAATCC TAAAAGTGTA ACTCTCTTTG |
| | GAGAAAGTGC AGGAGCAGCT TCAGTTAGCC TGCATTTGCT |
| | TTCTCCTGGA |
| | S H P L F T R A I L Q S G |
| | S S N A P W A V T S L Y E |
| | A R N R |
| 721 | AGCCATCCAT TGTTCACCAG AGCCATTCTA CAAAGTGGAT |
| | CCTCTAATGC TCCTTGGGCA GTAACATCTC TTTATGAAGC |
| | TAGGAACAGA |
| | T L T L A K L T G C S R D |
| | N E T E I V K C L R N K D |
| | P H E I |
| 811 | ACATTGACCT TGGCTAAATT GACTGGTTGC TCTAGAGATA |
| | ATGAGACTGA AATAGTCAAG TGCCTTAGAA ATAAAGATCC |
| | CCACGAAATT |
| | L L N E A F V V P Y G T L |
| | L S V N F G P T M D G D F |
| | L T E M |
| 901 | CTTCTGAATG AAGCATTTGT TGTCCCCTAT GGGACTCTCT |
| | TGTCAGTAAA CTTCGGTCCA ACCATGGATG GTGATTTTCT |
| | CACTGAAATG |
| | P D I L L E L G Q F K K T |
| | Q I L V G V N K D E G T A |
| | F L V Y |
| 991 | CCAGACATAT TACTTGAACT TGGACAATTT AAAAAAACCC |
| | AGATATTGGT GGGTGTTAAT AAAGATGAAG GGACAGCTTT |
| | TTTAGTCTAT |
| | G A P G F S K D N D S I I |
| | T R N E F Q E G L K I F F |
| | P G V S |
| 1081 | GGTGCTCCTG GCTTCAGCAA AGATAACGAT AGTATCATAA |
| | CTAGAAACGA ATTTCAGGAA GGTTTAAAAA TATTTTTTCC |
| | AGGCGTGAGT |
| | E F G K E S I L F H Y T D |
| | W V D D Q R P E N Y R E A |
| | L D D V |
| 1171 | GAGTTTGGAA AGGAATCCAT CCTTTTTCAT TACACAGACT |
| | GGGTAGATGA TCAGAGACCT GAAAACTACC GTGAGGCGTT |
| | GGATGATGTT |
| | V G D Y N I I C P A L E F |
| | T K K F S E W G N N A F F |
| | Y Y F E |
| 1261 | GTTGGGGATT ATAATATCAT ATGCCCTGCC TTGGAGTTTA |
| | CCAAGAAGTT CTCAGAATGG GGAAATAATG CCTTTTTCTA |
| | CTATTTTGAA |
| | H R S S K L P W P E W M G |
| | V M H G Y E I E F V F G L |
| | P L E R |
| 1351 | CACCGATCCT CCAAACTTCC GTGGCCAGAA TGGATGGGAG |
| | TGATGCATGG CTATGAAATT GAATTTGTCT TTGGGTTACC |
| | TCTGGAAAGA |
| | R V N Y T K A E E I L S R |
| | S I V K R W A N F A K Y G |
| | N P N G |
| 1441 | AGAGTTAATT ACACAAAAGC TGAGGAAATT TTGAGTAGAT |
| | CCATAGTGAA ACGGTGGGCA AATTTTGCAA AATATGGGAA |
| | TCCAAATGGG |
| | T H N N S T K W P V F K S |
| | T E Q K Y L T L N T E S S |
| | R I L T |
| 1531 | ACTCATAATA ATAGCACAAA ATGGCCTGTC TTCAAAAGCA |
| | CTGAACAAAA ATATCTAACC TTGAATACAG AGTCATCAAG |
| | AATATTGACT |
| | K L R A Q Q C R F W T S F |
| | F P K V L E M T G N I D E |
| | A E W E |
| 1621 | AAACTACGTG CTCAGCAATG CCGATTCTGG ACATCATTTT |
| | TTCCAAAAGT CTTGGAAATG ACAGGAAATA TTGATGAAGC |
| | AGAATGGGAG |
| | W K A G F H R W S N Y M M |
| | D W K N Q F N D Y T S K K |
| | E S C V |
| 1711 | TGGAAAGCAG GATTCCATCG CTGGAGCAAT TACATGATGG |
| | ACTGGAAAAA TCAATTTAAC GATTACACTA GCAAGAAAGA |
| | AAGTTGTGTG |
| | G L * |
| 1801 | GGTCTCTAA |

The longest open reading frame of RhBchE encoded a 574 amino acid polypeptide which had 95% sequence identity (96% sequence similarity) with HuBchE (gi:4557351) (SEQ ID NO:3), 91% identity (94% similarity) with rabbit BchE (gi:116354) (SEQ ID NO:4), 91% identity (95% similariTY) with horse BchE (gi:7381418) (SEQ ID NO:6), 88% identity (92% similarity) with cat BchE (gi:2981243) (SEQ ID NO:5), and 81% identity (89% similarity) with mouse BchE (gi:6857761) (SEQ ID NO:7). The polypeptide sequences of SEQ ID NOS:3-7 are shown below.

The polypeptide sequence of HuBchE (SEQ ID NO:3):

MHSKVTIICIRFLFWFLLLCMLIGKSHTEDDIIIATKNGKVRGMNLTVFGGTVTAFLGI
PYAQPPLGRLRFKKPQSLTKWSDIWNATKYANSCCQNIDQSFPGFHGSEMWNPNTDLSED
CLYLNVWIPAPKPKNATVLIWIYGGGFQTGTSSLHVYDGKFLARVERVIVVSMNYRVGAL
GFLALPGNPEAPGNMGLFDQQLALQWVQKNIAAFGGNPKSVTLFGESAGAASVSLHLLSP
GSHSLFTRAILQSGSFNAPWAVTSLYEARNRTLNLAKLTGCSRENETEIIKCLRNKDPQE
ILLNEAFVVPYGTPLSVNFGPTVDGDFLTDMPDILLELGQFKKTQILVGVNKDEGTAFLV
YGAPGFSKDNNSIITRKEFQEGLKIFFPGVSEFGKESILFHYTDWVDDQRPENYREALGD
VVGDYNFICPALEFTKKFSEWGNNAFFYYFEHRSSKLPWPEWMGVMHGYEIEFVFGLPLE
RRDNYTKAEEILSRSIVKRWANFAKYGNPNETQNNSTSWPVFKSTEQKYLTLNTESTRIM
TKLRAQQCRFWTSFFPKVLEMTGNIDEAEWEWKAGFHRWNNYMMDWKNQFNDYTSKKESC
VGL

The polypeptide sequence of rabbit BchE (SEQ ID NO:4):

MVTRSSHTEDVIITTKNGRIRGINLPVFGGTVTAFLGI
PYAQPPLGRLRFKKPQSLTKWSDIWNATKYANSCCQNIDQSFPGFHGSEMWNPNTDLSED
CLYLNVWIPTPKPKNATVMIWIYGGGFQTGTSSLQVYDGKFLTRVERVIVVSMNYRVGAL
GFLALPGNPEAPGNMGLFDQQLALQWVQKNIAAFGGNPKSVTLFGESAGAASVSLHLLSP
RSHPLFTRAILQSGSSNAPWEVMSLHEARNRTLTLAKFVGCSTENETEIIKCLRNKDAQE
ILLNEVFVVPFDSLLSVNFGPTVDGDFLTDMPDTLLQLGQLKKTQILVGVNKDEGTAFLV
YGAPGFSKDNTSIITRKEFQEGLKIFFPGVSEFGKESILFHYTDWVDEQRPENYREALDD
VVGDYNFICPALEFTKKFSEWGNNAFFYYFEHRSSKLPWPEWMGVMHGYEIEFVFGLPLE
RRVNYTKAEEILSRSIMKRWANFAKYGNPNGTQNNSTRWPVFKSTEQKYLTLNTESPRIY
TKLRAQQCRFWTLFFPKVLEMTGNIDEAEQEWKAGFHRWNNYMMAWKNHFNDYTSKKERC
AGF

The polypeptide sequence of cat BchE (SEQ ID NO:5):

MQSKGTIISIQFLLRFLLLWVLIGKSHTEEDIIITTKNGKVRGMNLPVLDGTVTAFLGI
PYAQPPLGRLRFKKPQFLTKWSDIWNATKHANSCYQNADQSFPGFPGSEMWNPNTDLSED
CLYLNVWSPTPKPKNATVMIWIYGGGFQTGTSSLPVYDGKFLARVERVIVVSMNYRVGAL
GFLALPGNPEIPGNMGLFDQQLALQWVQKNIAAFGGNPKSVTLFGESAGAGSVSLHLLSP
RSQPLFTRAILQSGSSNAPWAVMSLDEAKNRTLTLAKFIGCSKENDTEIIKCLRNKDPQE
ILLNELLVVPSDTLLSVNFGPVVDGDFLTDMPDTLLQLGQFKKTQILVGVNKDEGTAFLV
YGAPGFSKDNDSIITRKEFQEGLKIYFPGVSEFGREAILFYYVDLLDDQPAEKYREALDD
VLGDYNIICPALEFTTKESELGNNAFFYYFEHRSSQLPWPEWMGVMHGYEIEFVFGLPLE
RRVNYTRAEEILSRSIMNYWANFAKYGNPNGTQNNSTRWPAFRSTDQKYLTLNAESPKVY
TKLRAQQCRFWTLFFPKVLEMTGNIDEAEREWRAGFYRWNNYMMDWKNQFNDYTSKKESC
AGL

The polypeptide sequence of horse BchE (SEQ ID NO:6):

MQSWGTIICIRILLRFLLLWVLIGNSHTEEDIIITTKNGKVRGMNLPVLGGTVTAFLGI
PYAQPPLGRLRFKKPQSLTKWSNIWNATKYANSCYQNTDQSFPGFLGSEMWNPNTELSED
CLYLNVWIPAPKPKNATVMIWIYGGGFQTGTSSLPVYDGKFLARVERVIVVSMNYRVGAL
GFLALSENPEAPGNMGLFDQQLALQWVQKNIAAEGGNPRSVTLFGESAGAASVSLHLLSP
RSQPLFTRAILQSGSSNAPWAVTSLYEARNRTLTLAKRMGCSRDNETEMIKCLRDKDPQE
ILLNEVFVVPYDTLLSVNFGPTVDGDFLTDMPDTLLQLGQFKRTQILVGVNKDEGTAFLV
YGAPGFSKDNNSIITRKEFQEGLKIFFPRVSEFGRESILFHYMDWLDDQRAENYREALDD
VVGDYNIICPALEFTKKFSELGNDAFFYYFEHRSTKLPWPEWMGVMHGYEIEFVFGLPLE
RRVNYTKAEEILSRSIMKRWANFAKYGNPNGTQSNSTRWPVFKSTEQKYLTLNTESPKVY
TKLRAQQCRFWTLFFPKVLELTGNIDEAEREWKAGFHRWNNYMMDWKNQFNDYTSKKESC
SDF

The polypeptide sequence of mouse BchE (SEQ ID NO:7):

MQTQHTKVTQTHFLLWILLLCMPFGKSHTEEDFIITTKTGRVRGLSMPVLGGTVTAFLGI
PYAQPPLGSLRFKKPQPLNKWPDIHNATQYANSCYQNTDQAFPGFQGSEMWNPNTNLSED
CLYLNVWIRVPKPKNATVMVWIYGGGFQTGTSSLPVYDGKFLARVERVIVVSMNYRVGAL
GFLAFPGNPDAPGNMGLFDQQLALQWVQRNIAAFGGNPKSITIFGESAGAASVSLHLLCP
QSYPLFTRAILESGSSNAPWAVKHPEEARNRTLTLAKFTGCSKENEMEMIKCLRSKDPQE
ILRNERFVLPSDSILSINFGPTVDGDFLTDMPHTLLQLGKVKKAQILVGVNKDEGTAFLV
YGAPGFSKDNDSLITRKEFQEGLNMYFPGVSRLGKEAVLFYYVDWLGEQSPEVYRDALDD
VIGDYNIICPALEFTKKFAELENNAFFYFFEHRSSKLPWPEWMGVMHGYEIEFVFGLPLG
RRVNYTRAEEIFSRSIMKTWANFAKYGHPNGTQGNSTMWPVFTSTEQKYLTLNTEKSKIY
SKLRAPQCQFWRLFFPKVLEMTGDIDETEQEWKAGFHRWSNYMMDWQNQFNDYTSKKESC
TAL

Also provided is an alignment of RhBchE sequence of the invention (SEQ ID NO:8) to M62777 (partial RhBchE) (SEQ ID NO:9) and HuBchE (NCBI NM 000055) (SEQ ID NO:10).

The polynucleotide sequence encoding RhBchE (SEQ ID NO:8) is shown below.

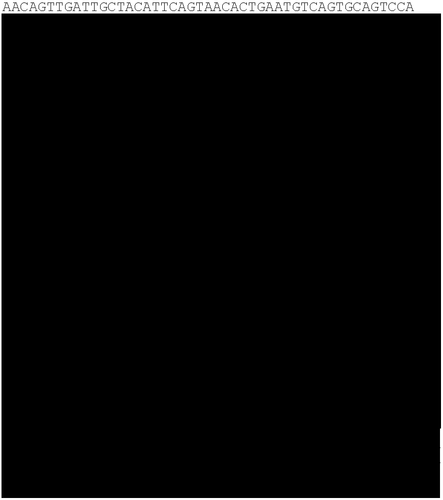

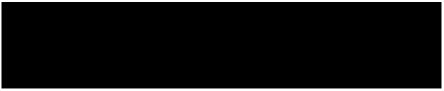

The polynucleotide sequence encoding the sequence of partial RhBchE (BV211040) (SEQ ID NO:9):

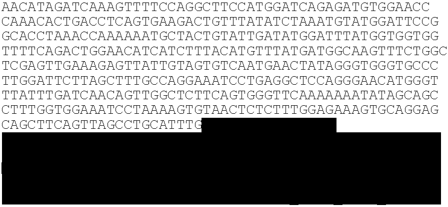

The polynucleotide sequence encoding HuBchE (SEQ ID NO:10):

AGTAACAGTTGATTGTTACATTCAGTAACACTGAATGTCAGTGCAGTCCA
ATTTACAGGCTGGAGCAGCAGCTGCATCCTGCATTTCCCCGAAGTATTAC
ATGATTTTCACTCCTTGCAAAC-TTTGCCATCTTTGTTGCAGAGAATCGG
AAATCAATATGCATAGCAAAGTCACAATCATATGCATCAGATTTCTCTTT
TGGTTTCTTTTGCTCTGCATGCTTATTGGGAAGTCACATACTGAAGATGA
CATCATAATTGCAACAAAGAATGGAAAAGTCAGAGGGATGAACTTGACAG
TTTTTGGTGGCACGGTAACAGCCTTTCTTGGAATTCCCTATGCACAGCCA
CCTCTTGGTAGACTTCGATTCAAAAAGCCACAGTCTCTGACCAAGTGGTC
TGATATTTGGAATGCCACAAAATATGCAAATTCTTCCTGTCAGAACATAG
ATCAAAGTTTTCCAGGCTTCCATGGATCAGAGATGTGGAACCCAAACACT
GACCTCAGTGAAGACTGTTTATATCTAAATGTATGGATTCCAGCACCTAA
ACCAAAAAATGCCACTGTATTGATATGGATTTATGGTGGTGGTTTTCAAA
ACCAAAAAATGCCACTGTATTGATATGGATTTATGGTGGTGGTTTTCAAA
CTGGAACATCATCTTTACATGTTTATGATGGCAAGTTTCTGGCTCGGGTT
GAAAGAGTTATTGTAGTGTCAATGAACTATAGGGTGGGTGCCCTAGGATT
CTTAGCTTTGCCAGGAAATCCTGAGGCTCCAGGGAACATGGGTTTATTTG
ATCAACAGTTGGCTCTTCAGTGGGTTCAAAAAAATATAGCAGCCTTTGGT
GGAAATCCTAAAAGTGTAACTCTCTTTGGAGAAAGTGCAGGAGCAGCTTC
AGTTAGCCTGCATTTGCTTTCTCCTGGAAGCCATTCATTGTTCACCAGAG
CCATTCTGCAAAGTGGATCCTTTAATGCTCCTTGGGCGGTAACATCTCTT
TATGAAGCTAGGAACAGAACGTTGAACTTAGCTAAATTGACTGGTTGCTC
TAGAGAGAATGAGACTGAAATAATCAAGTGTCTTAGAAATAAAGATCCCC
AAGAAATTCTTCTGAATGAAGCATTTGTTGTCCCCTATGGGACTCCTTTG
TCAGTAAACTTTGGTCCGACCGTGGATGGTGATTTTCTCACTGACATGCC
AGACATATTACTTGAACTTGGACAATTTAAAAAAACCCAGATTTTGGTGG
GTGTTAATAAAGATGAAGGGACAGCTTTTTTAGTCTATGGTGCTCCTGGC
TTCAGCAAAGATAACAATAGTATCATAACTAGAAAAGAATTTCAGGAAGG
TTTAAAAATATTTTTTCCAGGAGTGAGTGAGTTTGGAAAGGAATCCATCC
TTTTTCATTACACAGACTGGGTAGATGATCAGAGACCTGAAAACTACCGT
GAGGCCTTGGGTGATGTTGTTGGGGATTATAATTTCATATGCCCTGCCTT
GGAGTTCACCAAGAAGTTCTCAGAATGGGGAAATAATGCCTTTTTCTACT
ATTTTGAACACCGATCCTCCAAACTTCCGTGGCCAGAATGGATGGGAGTG
ATGCATGGCTATGAAATTGAATTTGTCTTTGGTTTACCTCTGGAAAGAAG
AGATAATTACACAAAAGCCGAGGAAATTTTGAGTAGATCCATAGTGAAAC
GGTGGGCAAATTTTGCAAAATATGGGAATCCAAATGAGACTCAGAACAAT
GAATACAGAGTCAACAAGAATAATGACGAAACTACGTGCTCAACAATGTC
GATTCTGGACATCATTTTTTCCAAAAGTCTTGGAAATGACAGGAAATATT
GATGAAGCAGAATGGGAGTGGAAAGCAGGATTCCATCGCTGGAACAATTA
CATGATGGACTGGAAAAATCAATTTAACGATTACACTAGCAAGAAAGAAA
GTTGTGTGGGTCTCTAATTAATAGATTTACCCTTTATAGAACATAT-TT
TCCTTTAGATCAAGGCAAAAATATCAGGAGCTTTTTTACACACCTACTAA
AAAAGTTATTATGTAGCTGAAACAAAAATGCCAGAAGGATAATATTGATT
CCTCACATCTTT-AACTTAGTATTTTACCTAGCATTTCAAAACCCAAATG
GCTAGAACGTGTTTAATTAAATTTCACAATATAAAGTTCTACAGTTAATT

RhBchE residues that are of interest for comparison among different species are shown in Table 1 below.

TABLE 1
Residues of interest in RhBchE as compare to enzymes from other
species.

| | |
|---|---|
| Residues in RhBchE that are | L-17 (F), VS (I), L21 (F), Y66 (C), |
| different from HuBchE | Ml10(L), P215 (S), S227 (F), T245 (N), |
| | D255 (E), V261 (1), H270 (Q), L285 (P), |
| | M294 (V), E301 (D), D342 (N), N348 |
| | (K), D390 (G), 1398 (F), V454 (D), |
| | G482 (E), H484 (Q), K489 (5), 5508 |
| | (T), L511(M), and S551 (N) |
| Residues in RhBchE differ | L-17 (F), V5 (I), L21 (F), Y66 (C), |
| yet conserved with HuBchE | M110 (L), T245 (N), D255 (E), V261 (I), |
| | H270 (Q), L285 (P), M294 (V), E301 (D), |
| | 1398 (F), H484 (Q), S508 (T), L511 (M), |
| | and S551 (N) |
| Residues in RhBchE that | P215 (S), S227 (F), D342 (N), D390 (G), |
| are not conserved with | V454 (D), G482 (E), K489 (S |
| HuBchE but conserved with | |
| other animal BchE | |
| Residues in RhBchE not | N348(K) |
| conserved with any other | |
| species | |
| Residues conserved between | A7(T), T19(P), G212(RIQ), E308(Q), |
| HuBchE and RhBchE but not | L511(Y), S524(L) |
| conserved with other species | |

Compared to the sequence of HuBchE, RhBchE contains 25 residues with different amino acids. Among these 25 different residues, 17 amino acids are conserved as similar amino acids. When compared to the BchE enzyme amino acid sequences from different animal species, including human, rabbit, cat, horse, and mouse, RhBchE has the highest similarity to HuBchE. There are six residues that are conserved between HuBchE and RhBchE but are not conserved with other species. However, seven out of the eight non-conserved amino acids (P215, S227, D342, D390, V454, G482, and K489) in RhBchE as compared to HuBchE are actually conserved with the other animal enzymes. The only amino acid residue that is specific for RhBchE is N348, whereas enzymes from all other species listed have Lys at this position (Table 1).

### EXAMPLE 7

### Expression of Functional Recombinant RhBchE

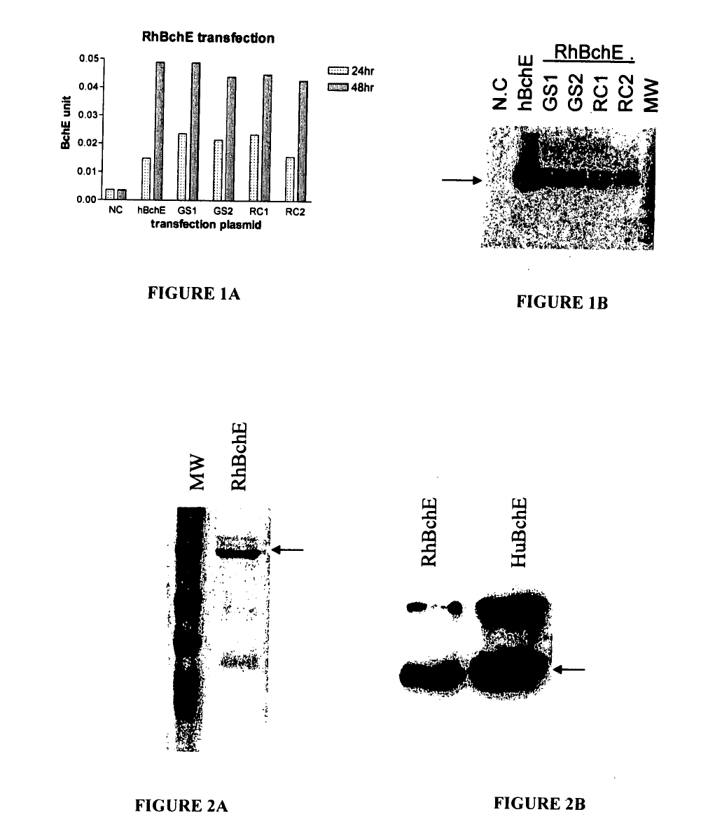

Expression of recombinant RhBchE was examined in both transient transfected and stably transfected CHO cells. Activity of expressed recombinant BchE enzyme was monitored from assay medium for BchI hydrolysis using the Ellman reaction. The expression of BchE protein was also confirmed by western blot analysis, indicating that the recombinant RhBchE recognized by the anti-HuBchE polyclonal antibody and co-migrated with recombinant HuBchE (FIG. 1).

Purification of RhBchE from M. mulatta serum. After three steps of chromatography, the RhBchE was purified to approximately 70% purity based on SDS-PAGE and commassie blue staining (FIG. 2A). Western blot analysis with anti-HuBchE polyclonal antibody showed that the antibody recognized the RhBchE very efficiently and the immuno-reactive band co-migrated with purified HuBchE (FIG. 2B). Pegylation or other derivatizations of BchE can be done to improve the stability of any protein identified in the invention described herein.

Substrate specificity of RhBchE. Enzyme fractions purified, as described in Example 1, were analyzed for BchI hydrolysis assay using the Ellman method (Table 2). Table 2 demonstrates kinetic constants for purified RhBchE and HuBchE determined in 50 mM potassium phosphate, pH 7.4, at 30° C. for butyrylthiocholine and (−)-cocaine.

TABLE 2
Kinetic constants for purified RhBchE and HuBchE.

| | Butyrylthiocholine iodide | |
|---|---|---|
| | Kₘ, μM | k_{cal} min⁻¹ |
| RhBchE | 71.1 ± 8.4 | 4.75 × 10³ ± 0.35 × 10³ |
| HuBchE | 26.1 ± 5.2 | 14.05 × 10³ ± 2.19 × 10³ |

As shown in Table 2-4, the RhBchE showed a significant difference with HuBchE in substrate specificity. While the RhBchE had a 2.7-fold lower binding affinity to BchI (Km 71.1 μM), the affinity to (−)-cocaine was about 2.9-fold higher (Kᵢ=4.7 μM) than HuBchE (Kᵢ=13.6 μM).

The substrate specificity of RhBchE was tested and cocaine hydrolytic products tested as inhibitors of the enzyme activity, the Kᵢ value for different compounds chemically related to cocaine were determined, as shown in Table 3 (BchI hydrolysis inhibition in 50 mM potassium phosphate, pH 7.4, at 25° C.).

TABLE 3
Kᵢ values of cocaine and related compounds on BchI hydrolysis
inhibition.

| | Monkey BchE | Human BchE |
|---|---|---|
| (−)-cocaine | 4.7 μM | 13.6 μM |
| (+)-cocaine | 15.3 μM | 8.0 μM |
| (−)-norcocaine | 119 μM | 43 μM |

As seen from Table 3, both (+)-cocaine and (−)-norcocaine has a ˜2 fold higher Kᵢ for RhBchE than HuBchE, while (−)-cocaine has a ˜3 fold lower Kᵢ for RhBchE than HuBchE, suggesting that the RhBchE is more structurally selective for (−)-cocaine than other substrates. (−)-Ecgonine methyl ester did not inhibit BchI hydrolysis at the concentration tested (0-100 μM).

Table 4 shows the kinetic constants for RhBchE and HuBchE for (−) cocaine (Xie et al., 1999; Mets et al., 1998) and shows that RhBchE has an improved k_{cat}/Kₘ compared to previously reported cocaine hydrolysis catalysts and HuBchE mutants.

TABLE 4
Kinetic constants for RhBchE and HuBchE for (−) cocaine.

| | | | k_{cal}/Kₘ |
|---|---|---|---|
| Native BchE | k_{cal}(min⁻¹) | Kₘ(μM) | (μM⁻¹ min⁻¹) |
| Monkey | 22.3 | 6.7\| | 3.33 |
| Human | 2.4 | 10.6 | 0.23 |
| hBchE¹ | 3.9 | 14 | 0.28 |
| A328Y hBchE¹ | 10.2 | 9 | 1.13 |
| A328F hBchE¹ | 5.8 | 24 | 0.24 |
| ISAIO mAb² | 2.3 | 220 | 0.01 |

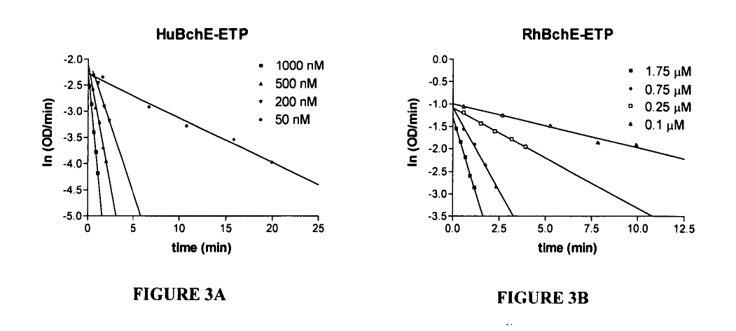

The catalytic efficiency (k_{cat}/Kₘ) for cocaine hydrolysis for RhBchE is over 10-fold greater than the human counterpart. When RhBchE was compared with HuBchE for its interaction with OP, the inhibition of RhBchE and HuBchE by ETP was first-order (FIG. 3). A re-plot of the kₐₚₚ first order rate constant (for inhibition) versus the concentration of ETP resolved the inhibition constant for the reaction of RhBchE and HuBchE with ETP. The difference in sensitivity between RhBchE and HuBchE is shown in Table 5 (inhibition rate constants for ETP were obtained through double reciprocal plot of kₐₚₚ (min⁻¹) using ETP concentrations presented in FIG. 3).

TABLE 5
Inhibition rate constants for ETP.

| | HuBchE | RhBchE |
|---|---|---|
| kᵢ × 10³ (M⁻¹ min⁻¹) | 1860 ± 135 | 764.4 ± 40.5 |

### EXAMPLE 8

### OP Analogue Synthesis and Inhibition of HuBchE

OP analogues mimicking the structures of sarin, soman, and VX respectively, were successfully synthesized as described in Examples 12 and 13. The OP analogues were tested for their interaction with HuBchE and the G117H/E197Q HuBchE. The results are shown in Table 6 (HuBchE and G117H/E197Q HuBchE were incubated with 0.5 mM of indicated compounds or control buffer for 48 hr at 4° C.).

TABLE 6
OP Inhibition of WT and G117H/E197Q HuBchE.

| | G117H/E197Q | WT |
|---|---|---|
| Compound 1 | 74.30% | 1.23% |
| Compound 2 | 74.73% | 1.93% |
| Compound 3 | 55.05% | 0.06% |
| Echothiophate | 12.77% | 0.02% |
| buffer | 100.00% | 100.00% |

As shown in Table 6, WT HuBchE was inhibited by all three compounds with less than 2% remaining activity. The remaining enzyme activity was examined by Ellman reaction using 1 mM BchI. However, the G117H//E197Q HuBchE variant still retained over 50% activity for all three compounds. This finding is in good agreement with earlier reports of the G117H/E197Q enzyme variant being resistant to OP inhibition, indicating that the three OP analogues synthesized are interacting with the HuBchE enzymes similarly as other OP compounds previously examined. In addition, when we examined the BchI hydrolysis for longer periods of time, the WT remaining activity post incubation with compounds 1 and 2 was significant, while no detectable activity was observed for the WT enzyme when compound 3 or echothiophate was present in the incubation. The resistance of G117H/E197Q HuBchE to OP compound inhibition presented here indicated an important feature that can be implemented in the functional screening design. In principle, this approach (replacing an oxygen ester with a sulfur) can be applied to any ester to develop a HT functional screen. Additional examples can be seen in Example 13.

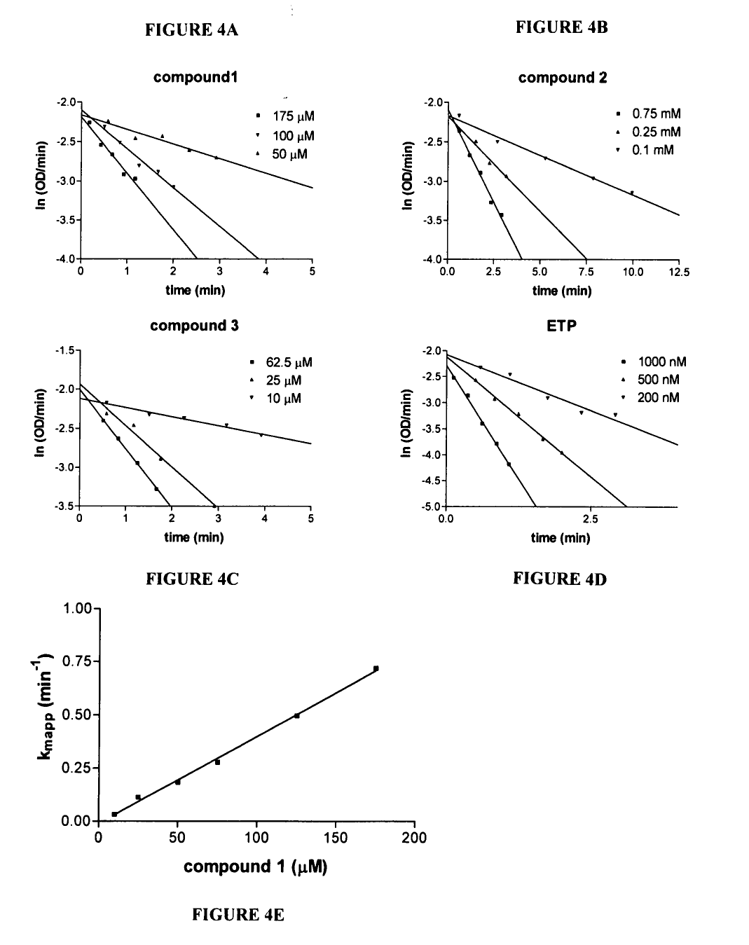

Inhibition rate constant determination for inhibition of HuBchE by the model OP compounds. The kinetics for time-dependent inhibition of purified HuBchE by the model OP compounds was studied with seven inhibitor concentrations and five time points for each inhibitor concentration. The inhibition of HuBchE by all OP model compounds was first-order (FIG. 4A shows selected inhibitor concentrations for compound 1, 2, 3 and echothiophate). A re-plot of the kₐₚₚ (first order rate constant for inhibition) versus the concentration of the OP compound resolved the inhibition constant for the reaction of HuBchE with the tested OP compounds (Table 7). Inhibition constants of OP compounds for HuBchE were obtained through double reciprocal plots of kₐₚₚ (min⁻¹) versus OP concentration shown in FIG. 4. FIG. 4B shows a representative plot from compound 1.

TABLE 7
Inhibition constants of OP compounds for huBchE.

| | Echo- | | | |
|---|---|---|---|---|
| | thiophate | Compound 1 | Compound 2 | Compound 3 |
| kᵢ × 10³ | 1860 ± 135 | 4.1 ± 0.12 | 1.73 ± 0.42 | 34.4 ± 0.93 |
| (M⁻¹min⁻¹) | | | | |
| k₂ (min⁻¹) | n.d. | >0.8 | 0.62 ± 0.047 | 1.04 ± 0.16 |
| K_{d} (μM) | n.d. | >200 | 358.7 ± 85.7 | 30.3 ± 11.8 |

Compound stability in culture medium and spontaneous hydrolysis. Because the OP model compounds are used for cell-based assays and due to the expected slow turn-over rate and long incubation periods expected, it is important that these compounds be stable under the assay conditions in the presence of culture medium. The enzyme independent spontaneous hydrolysis (i.e., buffer mediated-, H₂O mediated-, and medium-mediated hydrolysis) of the compounds was monitored by incubation of compounds in assay buffer in the presence of DTNB without enzyme followed by UV-Vis at 412 nm. All three compounds showed a non-detectable hydrolysis, indicating that no spontaneous hydrolysis (or very limited background hydrolysis) of these compounds was detected under the assay conditions used for functional screening. Compound stability was also examined by the ability to inhibit HuBchE before and after long-term storage in H₂O at room temperature and then tested for their capability to inhibit HuBchE. The results indicated that compound 1 and 2 were fairly stable with the storage conditions: there was only a slight decrease in ability to inhibit the HuBchE activity. However, compound 3 was not indefinitely stable under the conditions.

### EXAMPLE 9

### Gene Shuffle to Create RhBchE/HuBchE Chimeras

We have shown that RhBchE has over 10-fold greater cocaine hydrolysis activity than HuBchE. There is a difference in 23 amino acids between RhBchE and HuBchE. The amino acid residues from eight randomly selected clones that are different between HuBchE and RhBchE are shown in Table 8 below.

TABLE 8
The Amino Acid Residue Difference between HuBchE or RhBchE.

| aa # | huBchE | RhBchE | Clone1 | Clone2 | Clone3 | Clone4 | Clone5 | Clone6 | Clone7 | Clone8 |
|---|---|---|---|---|---|---|---|---|---|---|
| 47 | V | V | V | V | G | V | V | V | V | V |
| 48 | F | L | F | F | F | F | F | F | F | F |
| 54 | T | T | A | T | T | T | T | T | T | T |
| 90 | A | A | A | T | A | A | A | A | A | A |
| 94 | C | Y | Y | Y | Y | Y | C | C | C | Y |
| 138 | L | M | M | M | M | L | M | M | L | M |
| 155 | V | X | V | V | V | V | V | V | V | V |
| 186 | G | G | R | G | G | G | G | G | G | G |
| 243 | S | P | P | S | P | S | S | S | P | P |
| 255 | F | S | F | F | F | S | F | F | S | S |
| 273 | N | T | N | N | N | T | N | T | T | T |
| 283 | E | D | E | D | D | D | E | E | D | E |
| 285 | E | E | E | G | G | G | E | E | G | E |
| 289 | I | V | I | V | V | I | I | I | V | I |
| 298 | Q | Q | Q | H | H | Q | Q | Q | Q | Q |
| 313 | P | L | P | L | L | P | L | L | P | P |
| 322 | V | M | V | V | V | V | M | V | V | V |
| 329 | D | E | E | D | D | E | E | D | D | D |
| 370 | N | D | D | D | N | N | D | N | D | D |
| 376 | K | N | N | N | N | K | K | N | N | N |
| 402 | T | T | T | T | T | I | T | T | T | T |
| 405 | V | V | I | V | I | I | V | V | V | I |
| 418 | G | D | D | D | D | G | G | G | D | D |
| 422 | G | G | G | G | G | G | G | R | G | G |
| 426 | F | I | I | F | I | F | F | F | F | I |
| 480 | R | R | G | R | G | G | R | G | G | R |
| 482 | D | V | V | D | V | V | D | V | D | D |
| 486 | E | E | G | E | E | G | E | G | E | E |
| 494 | S | S | S | S | P | S | S | S | S | S |
| 495 | I | I | T | T | I | T | T | T | I | T |
| 510 | E | G | G | E | E | G | G | G | E | E |
| 512 | Q | H | H | Q | Q | Q | H | H | Q | Q |
| 517 | S | K | K | S | S | S | K | K | S | S |
| 523 | S | N | N | S | S | S | S | S | S | S |
| 525 | E | E | E | K | E | E | E | E | E | E |
| 536 | T | S | S | T | T | T | S | S | T | T |
| 539 | M | L | L | M | M | M | L | L | L | M |
| 564 | I | I | I | I | I | I | V | I | I | I |
| 575 | F | F | F | F | F | F | F | S | F | F |
| 577 | R | R | R | R | H | R | R | R | R | R |
| 579 | N | S | S | S | S | S | S | N | N | S |

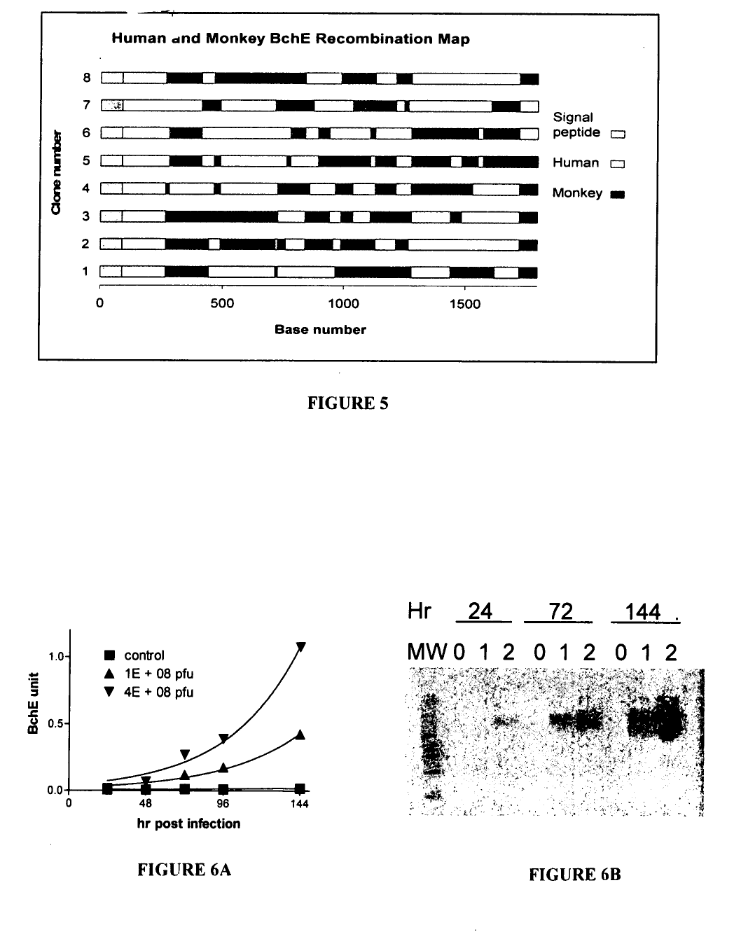

It is believed that some, if not all, of these variants contribute to improved cocaine or other ester hydrolysis activity for RhBchE. In order to identify the best combination of selected beneficial variations, a library of chimeras from the HuBchE and RhBchE was created. DNA shuffling technology described above was used. Plasmid DNAs were prepared from eight randomly selected clones. The BchE coding region from the eight clones were sequenced and analyzed. FIG. 5 depicts the eight recombined chimeras from HuBchE and RhBchE. The 5′ region of all clones encode for the HuBchE signal peptide. Multiple recombination events (averaging about 12 crossovers for each clone) occurred for the 8 randomly selected clones at different sites. This indicated that these eight clones represented a library of highly diversified chimeras of HuBchE and RhBchE recombinants. This diversified library was the basis for functional selection. The recombinant process also introduced a low level of point mutations. Out of the eight sequenced clones, 12 novel amino acid mutations were identified. No frame-shift or premature termination mutations were found in these sequences. Such a low mutation rate is ideal because it increases the diversity of the library and at the same time does not introduce too many deleterious mutations which could lead to non-functional enzymes.

### EXAMPLE 10

### AD-Mediated Expression of HuBchE

The AD system is another viral expression system that has been used extensively to express human proteins. AD can infect a broad range of mammalian cells and permit expression of recombinant proteins in many dividing and non-dividing mammalian cell lines. Medium from cells infected with various titers of the recombinant virus are analyzed for BchE activity, and show that BchE activity in the culture medium showed dose-dependent and time-dependent expression of HuBchE post-infection of COS cells. Similar results were obtained with CHO cells (data not shown). As shown in FIG. 6, accumulated BchE activity in culture medium increased exponentially over time. The AD construct was replication-incompetent; no additional viral particles can be produced when COS cells are infected. Therefore the continuous increase of enzyme activity in the culture medium indicated the infected cells continuously produced the BchE enzyme over days. When examined by microscope, CPE (cell round up) caused by the AD infection was not apparent until day 4 and 5 post infection. FIG. 6B showed that western blot analysis of the culture medium reflected the presence of HuBchE in a time-dependent and dose-depended manner and was in good agreement with results shown in FIG. 6A.

Construction of the Adenoviral expression vector for WT and G117H/E197Q HuBchE. The Viralpower AD Expression system was used to clone the HuBchE enzyme. The cloning and recombination efficiency of the construction procedure used for library construction was determined. Using 300 ng pAD/CMV/V5/DEST plasmids in the recombination reaction and using chemical competent cells with a transformation efficiency of 1×10⁶ cfu/μg, we obtained approximately 50 recombinant colonies for each recombination reaction. Restriction digestion of 12 randomly selected colonies revealed that 75% carried the correct HuBchE insert. This shows that for cloning of the mutation library, using 10 μg of DNA for the recombination step, and more efficient electrocompetent cells (1×10¹⁰ cfu/μg) can generate 1×10⁶ recombination clones. This proves the feasibility of using the AD construct to generate a large mutation library efficient for successful functional screening.

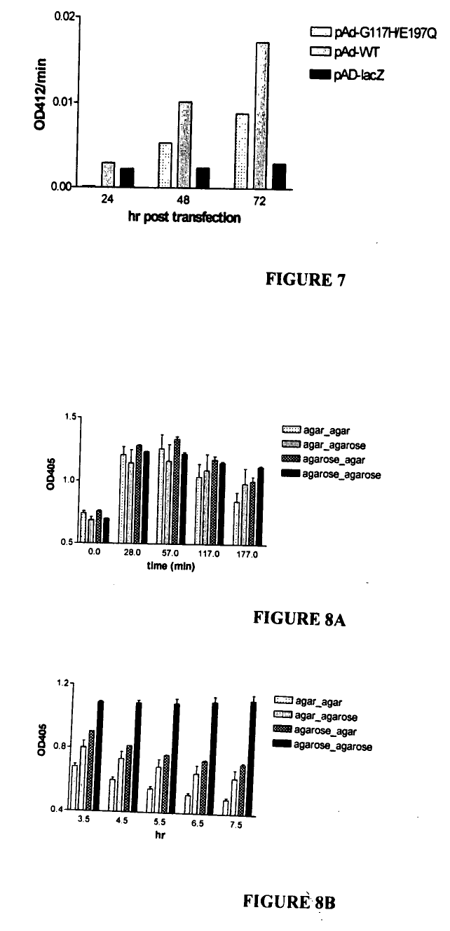

Generation of HuBchE-AD and validation expression of recombinant WT and G117H/E197Q HuBchE with the AD expression system. Recombinant WT- and G117H/E197Q-hBchE-AD were generated as described in Example 4 and collected. FIG. 7 shows that transfection of pAD-WT-hBchE and pAD-G117H/E197Q-hBchE resulted in increasing the HuBchE activity while transfection of the control vector pAD-lacZ only showed consistent background activity. This indicated that construction of the pAD-WT-hBchE and pAD-G117H/E197Q-hBchE was successful.

Validation of functional screening technology for highly efficient OP detoxication agents. Developing a solid-phase cell-based enzyme assay has significant advantages over assays using other enzyme assays in medium because of the ability to handle relatively large mutant libraries with less labor and cost. CHO-K1 cells stably expressing the HuBchE WT or G117H/E197Q HuBchE were first used to establish the assay conditions. The preseeded cells were washed and then overlaid with 1% agar in colorless MEM. After the agar solidified, the cells were returned to the incubator (overnight) to allow the secretion of the BchE enzyme and diffusion of the enzyme into agar. Based on observations under microscopy, the cells remained healthy for at least 10 days under such incubation conditions. Ellman reaction mixture containing 1 mM BchI and 0.1 mM DTNB were prepared in 1% agar in colorless MEM. The reaction mixture was overlaid on cell culture plates prepared above and incubated at room temperature. Visually detectable yellow color from the Ellman reaction was developed and the plates were scanned after 10 min incubation. The plate was also measured for OD405 absorption with a plate reader (Victor 2, Perkin Elmer) after 40 min incubation (data not shown). This experiment indicated that the cell-expressed HuBchE can diffuse into the agar and allow visually detectable yellow product to accumulate when provided with the Ellman reaction reagent.

Comparison of agar and agarose as the solid-phase media. During the above assays, even though we detected the hydrolysis of BchE via yellow color formation easily, we found that the yellow product was not indefinitely stable and disappeared upon long term incubation (several hours to overnight). Because we expected longer incubation time for some ester substrates such as OP compounds, we needed to address the stability issue. As shown in FIG. 8, the formation of the product in the first 30-60 min was not affected by using either agar or agarose in the two layers of coating, suggesting the HuBchE was produced and diffused into the solid phase similarly. However, over longer incubation periods, the presence of agar in the solid phase caused discoloration and reduction of the absorbance. Only when the cells were coated with agarose in both layers, was the product stabilized over long term incubation (FIG. 8B).

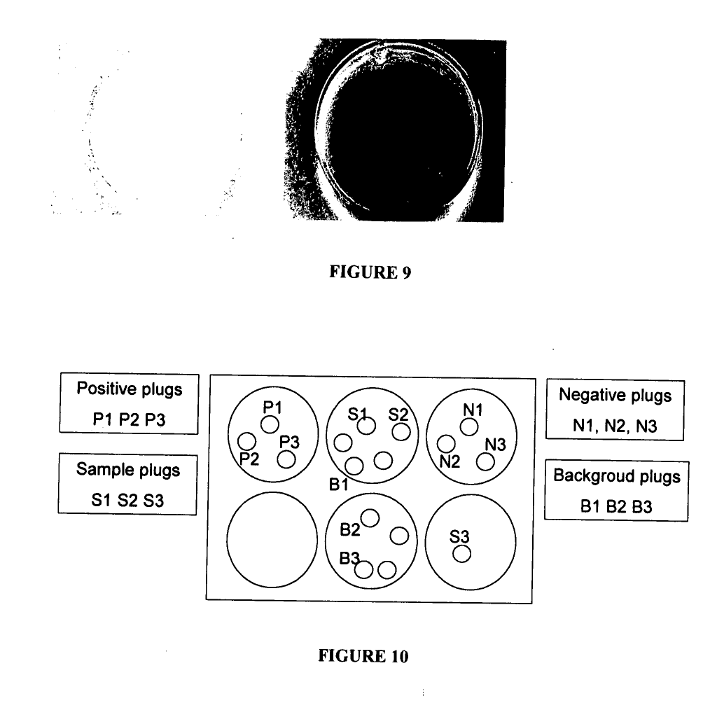

Localized detection of HuBchE activity. One advantage of developing solid-phase activity screening is the ability to identify functional activities in small localized areas. This allows convenient screening of large libraries in a cost-effective fashion. Using serial diluted CHO-K1 cells stably expressing WT HuBchE, we tested whether the currently the developed assay offered such an advantage. As shown in FIG. 9, visually detectable yellow spots corresponding to the localization of the HuBchE expressing cells became apparent within minutes after addition of substrate. For plates with more cell colonies (˜100 per plate), the spots were fused together and no longer distinguishable (data not shown). For plates with about 20 colonies per plate, the yellow spots were easily distinct (FIG. 9). The ability to distinctively identify ˜20 reactive spots on a 10 cm culture dish suggests that large libraries can be placed in culture plates as long as less than 20 positive clones are detected during the functional screening. The positive clones can then be easily purified after serial dilution.

### EXAMPLE 11

### Application of the Solid-Phase BchE Activity Assay for Detection of AD-BchE Recombinant Virus

We attempted to detect AD-recombinant virus using the solid-phase BchE activity assay. Using the AD system usually involves a slow tittering process due to slow plaque formation. The BchE activity assay allowed development of an alternative method for estimating virus titer in a shorter time frame. Serial-diluted HuBchE-AD virus was used to infect 293A cells pre-seeded in 6-well plate. An agarose-MEM mix was overlaid on cells at 1 hr post infection. Plates were stained for BchE activity in the following days after initial infection. While higher titer infected wells were stained yellow starting on the 2nd day post-infection, distinctive yellow spots similar to FIG. 9 appeared in low titer infected wells at 4 days post-infection. At day 4 post-infection, yellow spots developed within 30 min of application of BchI and DTNB. Spots were marked as they developed. In wells with more spots developed, the yellow color soon fused together. In wells with 4 or fewer spots, identification of the spot was easy. Using the assay developed here to substitute traditional plaque assays for virus titer determination saves about 7 days for each tittering process.

Virus was isolated from identified yellow spots, and plugs were cored-out from the agarose culture plate. FIG. 9 shows an illustration of specific plugs isolated during this experiment. Positive plugs were isolated from wells infected with high virus titers and showed yellow overall after BchE activity staining. Negative plugs were isolated from wells infected with highest virus dilutions with no yellow color showing after BchE activity staining. Sample plugs were isolated from wells with distinctive yellow spots developed after staining and the spots were cored as sample plugs. Background plugs were isolated from wells with distinctive yellow spots developed after staining and the non-stained area close to the spots were cored as background plugs. The isolated plugs were transferred to tubes containing 0.5 ml of cultured medium and incubated at 4° C. overnight. The medium from the positive plug and sample plug were diluted 10-fold. Medium incubated with plugs (N1 to 3, P1 to 3, S1 to 3, and B1 to 3) and the diluted medium (P′1 to 3 and S′1 to 3) were then used to infect 293A cells pre-seeded in 24-well plate. BchE activity in the medium was assayed at 24, and 48 hr post-infection and average activity was presented in Table 9.

TABLE 9
BchE activity of various plugs in the medium.

| Hr | N1-3 | B1-3 |
|---|---|---|
| 24.00 | 023 ± 0.04 | 0.36 ± 0.07 |
| 48.00 | 0.26 ± 0.03 | 2.06 ± 0.51 |
| 72.00 | 0.35 ± 0.05 | 6.47 ± 1.91 |
| Hr | P1-3 | P′1-3 |
| 24.00 | 1.36 ± 0.63 | 0.62 ± 0.31 |
| 48.00 | 4.14 ± 0.91 | 3.13 ± 1.17 |
| 72.00 | 6.52 ± 0.75 | 5.40 ± 0.34 |
| Hr | S1-3 | S′1-3 |
| 24.00 | 0.93 ± 0.09 | 0.32 ± 0.10 |
| 48.00 | 4.24 ± 0.30 | 2.34 ± 0.24 |
| 72.00 | 6.36 ± 1.29 | 6.11 ± 0.90 |

The negative plugs did not lead to BchE expression (N1-3). The positive and the sample plugs led to increasing BchE activity (S1-3 and P1-3). Interestingly, the background plugs isolated near the sample plugs resulted in low level of activity (B1-3, 48 hr). The activity level was lower than that obtained from 10-fold dilutions of the sample or positive plugs (B1-3, S′1-3, and P′1-3). This result indicated that 1) we can isolate HuBchE-AD by isolating yellow stained agarose plugs; 2) virus from the plugs diffused to a nearby area and can possibly cause less than 10% contamination for plugs close by (<0.5 cm). Plaques separated further apart will significantly improve the purity of the virus in each plug. These results have served as important reference points for Phase II functional screen design.

Validation of the functional screening assay. To validate the solid-phase functional screening and test the feasibility of using the solid phase assay to identify OP catalysts from the mutant libraries, the AD-G117/E197Q recombinant virus (10 pfu) was mixed with different amount of wild type HuBchE recombinant virus (0, 10, 100, and 500 pfu). The mixed recombinant viruses were used to infect 293A cells. 24 hr post infection, the cells were coated with 1% agarose in MEM. 48 hr post infection, 0.4 mM compound 1 was added to the coated cells to interact with expressed recombinant HuBchE and variant. At day 4 post infection, the coated cells were stained as described above using BchI as substrates in the presence of DTNB. A similar number of yellow spots was identified in plates infected with same number of AD-G117H/E197Q HuBchE virus mixed with increasing number of AD-WT HuBchE virus. The yellow agarose spots were cored out from the plate and incubated in serum-free medium in individual tubes. The recombinant viruses released in the medium were analyzed with PCR using AD specific primers that were across the gene insert. The PCR product was sequenced. Sequence analysis proved that for plugs isolated from the first 3 plates (with 10 pfu of G117H/E197Q and 0, 10, and 100 pfu of WT HuBchE), the isolated plug carried a clean G117H/E197Q sequence. For the plate with 10 pfu G117H/E197Q mixed with 500 pfu of WT HuBchE, a mixed sequence of G117H/E197Q and WT was observed. This experiment proved that with the AD expression system, the solid-phase assay can isolate compound 1-resistant HuBchE variants from a background of OP sensitive recombinant viruses. When background level recombinant virus level is high, plug purification step will improve the isolation efficiency.

Optimization and validation of the functional screening system with a site-saturation mutation library. The feasibility of the functional screening system was shown for the molecular evolution approach. In a high-throughput functional screening effort, the system is refined and fully validated for its sensitivity, throughput capacity, and reproducibility. To accomplish this, site-saturation mutagenesis is done at two amino acid positions G117 and E197. Site-saturation mutagenesis technology briefly described above was used. The final full length PCR product is sequenced and verified for the mutation codon incorporated. Plasmid DNAs are prepared from seven randomly selected pAD-huBuChE clones. The BchE coding region from the seven clones were sequenced and analyzed. The sequence results indicated that these seven clones represented a library of high diversity at amino acid positions 117 and 197. This diversified library serves as the basis for functional selection.

Functional screening. Based on results showed above, we designed the working flow chart (Scheme 12) of functional screening of OP catalytic enzyme using systems developed above. The functional screening involve steps of solid-phase screening, liquid-1-phase screening, plaque purification, activity confirmation, gene amplification, and sequencing. This designed work flow will allow the identification of BchE variants with desired organic or inorganic ester hydrolytic catalytic activity. A similar approach can be applied for isolation of cocaine catalytic enzyme.

In summary, we have cloned a novel BchE enzyme from M. mulatta and characterized the substrate specificity and inhibition kinetics. We synthesized three OP analogue compounds that are useful for functional screening for OP catalytic enzymes. We constructed mutation library and also validated a high level expression AD system for BchE functional screening. The solid-phase activity based functional screening was also developed and validated. Technologies and research tools established from this invention provided full capability of molecular evolution to continue improve HuBchE into a catalytic detoxification enzyme for nerve agents, cocaine, or other potentially harmful organic or inorganic esters

### EXAMPLE 12

### Chemical Synthesis of Novel OP Analogues

OP analogues were designed to mimic nerve agents VX, soman, and sarin, respectively.

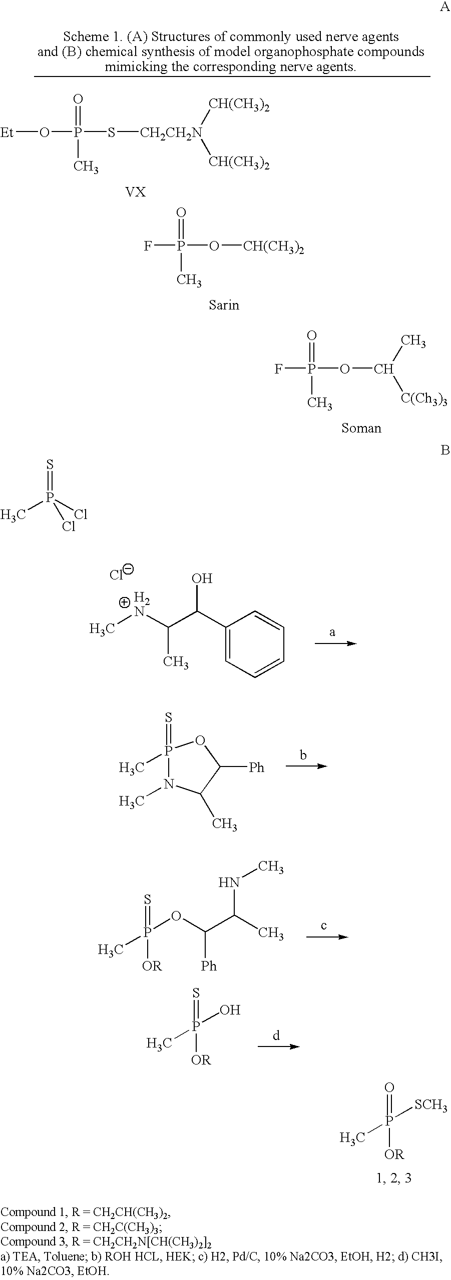

As illustrated in Scheme 1, the general procedure involves the reaction of (±)-ephedrine with methylphosphonothioic dichloride to form 2,3,4-trimethyl-5-phenyl-1,3,2-oxazaphospholidine-2-thione. The sequence then entails reaction of 2,3,4-trimethyl-5-phenyl-1,3,2-oxazaphospholidine-2-thione with an alcohol, and subsequent hydrogenolysis. The resulting alkyl hydrogen methylphosphonothioates are alkylated with iodomethane to afford the desired O-alkyl S-methyl methylphosphonothioate compounds. The chemical synthesis process of the target compound including characterization of all the intermediates and is presented below.

Synthesis of 2,3,4-trimethyl-5-phenyl-1,3,2-oxazaphospholidine-2-thione: A racemic mixture of 2,3,4-trimethyl-5-phenyl-1,3,2-oxazaphospholidine-2-thione was prepared according to the procedure of Cooper, et al (J. Chem. Soc. Perkin Trans. 1977, 17, 1969-80) (Scheme 2).

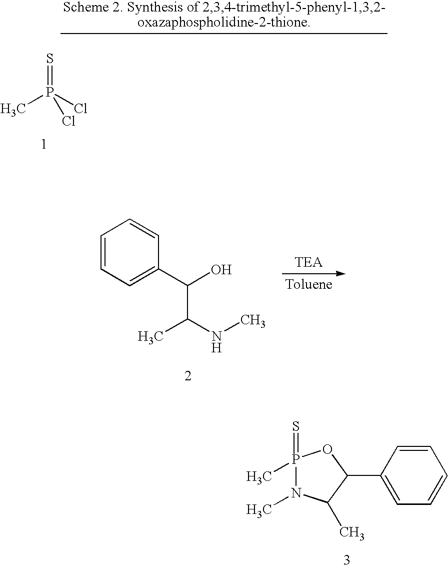

A solution of methylphosphonothioic dichloride (8.0 g, 53.7 mmol) in toluene (25 mL) was added slowly to a stirred solution of (±-ephedrine (13.0 g, 64.4 mmol) in triethylamine (27.2 g, 268 mmol) and toluene (210 mL). Once the addition was complete the reaction was stirred under argon, in the dark, at room temperature for 24 h, filtered, washed with water, dried (MgSO4), and concentrated in vacuo to afford a pale yellow oil. Column chromatography on silica (hexanes/ethyl acetate, 3:1, v/v) afforded a mixture of diasteromers as an oil (5.39 g, 22.4 mmol, 42%). TLC (hexanes/ethyl acetate, 3:1, v/v) RF=0.26; 1H NMR (CDCl3) 7.24-7.37 (m, 5 H), 5.64 (dd, JHP=3.0 Hz, JHH=6.0 Hz, 0.5 H), 5.47 (dd, JHP=2.1 Hz, JHH=5.7 Hz, 0.5 H), 3.61 (m, 1 H), 2.75 (d, JHP=12.3 Hz, 1.5 H), 2.66 (d, JHP=12.0 Hz, 1.5 H) 2.04 (d, JHP=14.4 Hz, 1.5 H), 1.93 (d, JHP=14.0 Hz, 1.5 H), 0.80 (d, JHP=6.6 Hz, 1.5 H), 0.73 (d, JHP=6.6 Hz, 1.5 H); MS (ESI) [M+H]+m/z calcd. for C11H18NOPS 242. found 242.

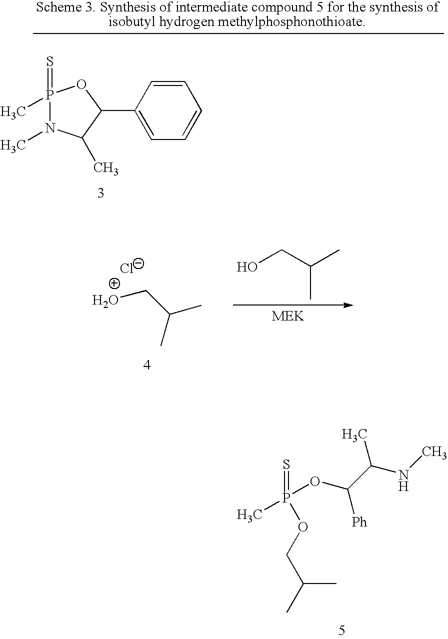

As shown in Scheme 3, a saturated solution of dry HCl in isobutyl alcohol (3 mL) and dry methyl ethyl ketone (MEK, 3 mL) was added slowly to a chilled, stirred solution of 2,3,4-trimethyl-5-phenyl-1,3,2-oxazaphospholidine-2-thione (1.05 g, 4.36 mmol) in isobutyl alcohol (7 mL) and MEK (7 mL). The reaction was stirred in the dark at room temperature for 1.5 h and then poured into ice-cold aqueous 10% Na2CO3 (25 mL). This aqueous mixture was directly used in the hydrogenation reaction without purification as described below.

### Synthesis of isobutyl hydrogen methylphosphonothioate

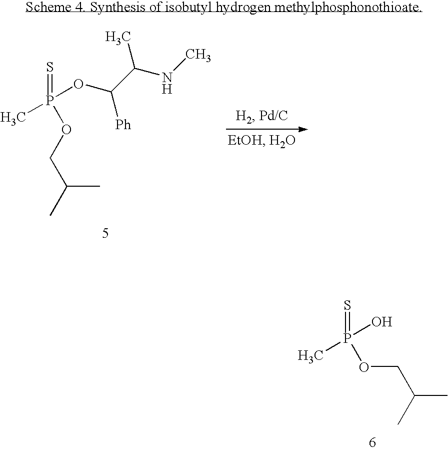

The crude mixture from the previous reaction containing 5 was diluted with water (50 mL) and ethanol (75 mL). To this solution was added Pd/C (160 mg) and then a balloon filled with H2(g) was fitted to the round bottom flask. After work-up and extraction with chloroform/isopropyl alcohol (4:1, v/v) the organic layers were combined, dried (MgSO4), filtered, and concentrated to afford a crude oil (390 mg, 53%). 1H NMR (CDCl3) 3.79 (m, 2 H), 1.91 (m, 1 H), 1.82 (d, JHP=15.6 Hz, 3 H) 0.90 (d, J=6.9 Hz, 6 H).

### Synthesis of O-Isobutyl S-methyl methylphosphonothioate

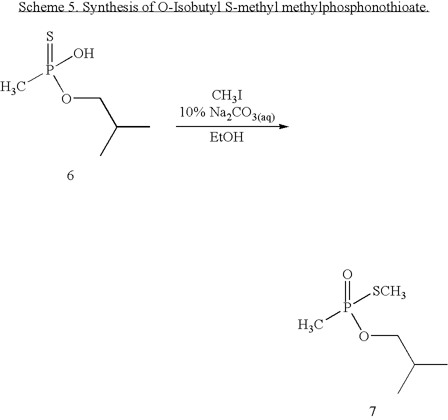

As shown in Scheme 5, to a solution of 6 (302 mg, 1.79 mmol) in 10% Na2CO3(aq) (2.5 mL) diluted with ethanol (25 mL), iodomethane (2.54 g, 17.9 mmol) was added. After 24 hr, the reaction was worked up and the organic material was washed with water, dried (MgSO4), and concentrated in vacuo to afford a light brown oil. The pure product was obtained by bulb-to-bulb distillation with a Kugelrohr apparatus to afford a clear oil (100 mg, 0.55 mmol, 31%). 1H NMR (CDCl3) 3.75 (m, 2 H), 2.21 (d, JHP=12.9 Hz, 3 H), 1.88 (m, 1 H), 1.71 (d, JHP=15.6 Hz, 3 H).

Synthesis of intermediate compound 9.

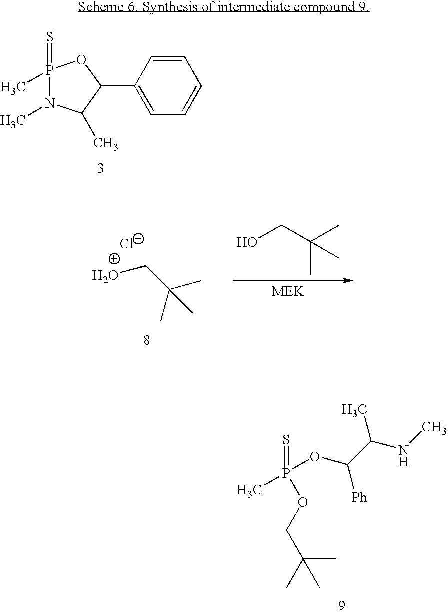

As shown in Scheme 6, a saturated solution of dry HCl in neopentyl alcohol (3.0 g, 34.0 mmol) and dry methyl ethyl ketone (MEK, 3 mL) was added slowly to a chilled, stirred solution of 2,3,4-trimethyl-5-phenyl-1,3,2-oxazaphospholidine-2-thione (1.05 g, 4.36 mmol) in neopentyl alcohol (7.0 g, 79.4 mmol) and MEK (7 mL). The reaction was stirred in the dark at room temperature for 1.5 h and then poured into ice-cold aqueous 100% Na2CO3 (25 mL). This aqueous mixture was directly used in the hydrogenation reaction without purification as described below.

### Synthesis of neopentyl hydrogen methylphosphonothioate

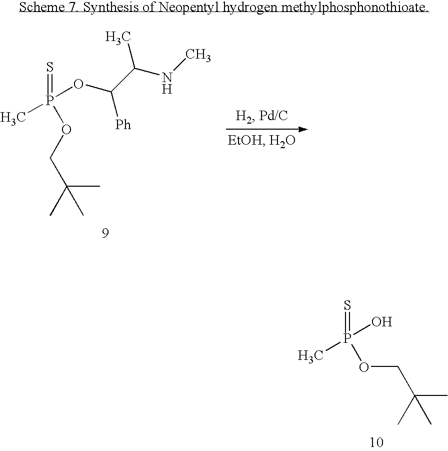

A crude mixture of 9 was diluted with water (50 mL) and ethanol (75 mL). To this solution was added Pd/C (181 mg) and then a balloon filled with H2(g) was fitted to the round bottom flask. After work-up, the organic material was combined, dried (MgSO4), filtered, and concentrated to afford a crude oil (700 mg, 3.84 mmol, 91%). 1H NMR (CDCl3) 3.6 (m, 2 H), 1.74 (d, JHP=15.6 Hz, 3 H), 0.89 (s, 9 H).

### Synthesis of O-Neopentyl S-methyl methylphosphonothioate

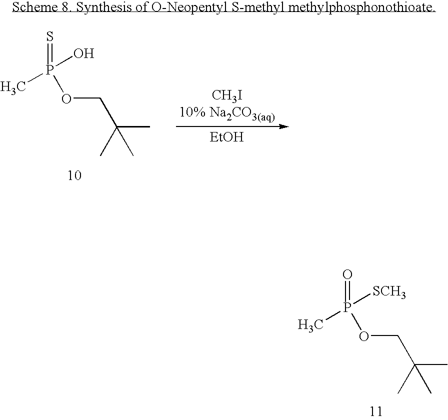

To a solution of 10 (700 mg, 3.84 mmol) in 10% Na2CO3(aq) (5.5 mL) diluted with ethanol (55 mL) was added iodomethane (5.45 g, 38.4 mmol). After 24 hr, the pure product was obtained by bulb-to-bulb distillation with a Kugelrohr apparatus to afford a clear oil (55 mg, 0.28 mmol, 7%). 1H NMR (CDCl3) 3.71 (m, 2 H), 2.29 (d, JHP=14.4 Hz, 3 H), 1.79 (d, JHP=15.6 Hz, 3 H), 0.95 (s, 9 H).

Synthesis of intermediate compound 13.

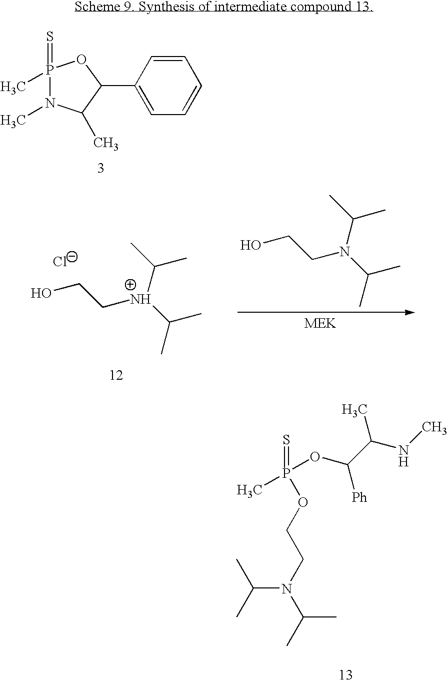

2-Diisopropylamino-ethanol hydrochloride salt (3.74 g, 20.6 mmol) was added to a chilled, stirred solution of 2,3,4-trimethyl-5-phenyl-1,3,2-oxazaphospholidine-2-thione (1.05 g, 4.36 mmol) in 2-diisopropylamino-ethanol (7.0 mL) and MEK (10 mL). The reaction was stirred in the dark at room temperature for 1.5 h and then poured into ice-cold aqueous 10% Na2CO3 (25 mL). This aqueous mixture was directly used in the hydrogenation reaction without purification as described below.

### Synthesis of 2-Diisopropylamino-ethyl hydrogen methylphosphonothioate

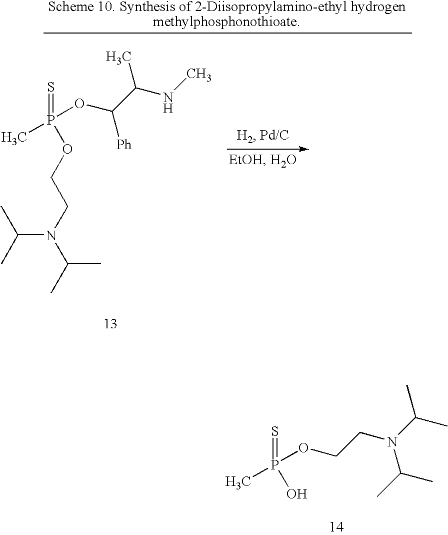

A crude mixture of 13 was diluted with water (50 mL) and ethanol (75 mL). To this solution was added Pd/C (172 mg) and then a balloon filled with H2(g) was fitted to the round bottom flask (Scheme 10). The flask was evacuated and then purged with hydrogen and stirred for 18 h. After work-up the organic material was combined, dried (MgSO4), filtered, and concentrated to afford a crude oil (505 mg, 2.11 mmol, 91%).

### Synthesis of O-2-Diisopropylamino-ethyl S-methyl methylphosphonothioate

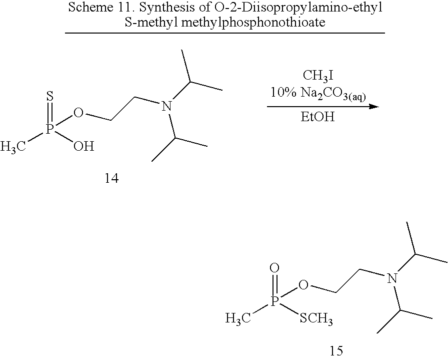

To a solution of crude 14 (505 mg, 2.11 mmol) in 10% Na2CO3(aq) (3.0 mL) diluted with ethanol (30 mL) was added iodomethane (5 g, 21.1 mmol) and stirred in the dark at room temperature for 24 h. After work-up, the organic layer afforded a light brown oil. The pure product was obtained by bulb-to-bulb distillation with a Kugelrohr apparatus to afford a clear oil (13 mg, 0.05 mmol, 2%). 1H NMR (CDCl3) 4.79 (m, 2 H), 3.99-4.21 (m, 4 H), 2.30 (d, JHP=13.2 Hz, 3 H), 1.75 (d, JHP=15.6 Hz, 3 H), 1.36 (d, J=6.0 Hz 6 H), 1.31 (d, J=6.3 Hz, 6 H).

### EXAMPLE 13

Chemical synthesis of nerve agent analogs of use in the Bioarray. The enantioselective synthesis of OPs to be used as analogs of nerve agents of use in procuring biomarkers, antibodies and of use in molecular evolution screening is described below. The compounds were designed to provide a similar phosphorylated ChE as the actual nerve agent OP but be a much less toxic analog. Thus, administration of the analogs give the same enzyme adducts.

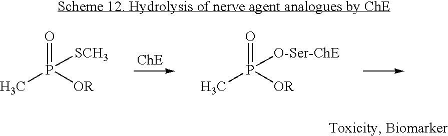

Due to the extreme toxicity of the target OP chemical warfare compounds, modification was designed for the nerve agent analogues to retain structural similarity yet possess reduced toxicity to allow for practical aspects of large-scale synthesis, handling and biological testing. We chemically synthesized the desired target compounds (i.e., 4-13 and racemic equivalents, 1-3) and have examined them as inhibitors of highly purified human butyrylcholinasterase (hBuChE). The results have validated our approach and shows that the potential for toxicity of the target compounds are significantly less than agents more directly related to nerve agents. This is useful from a practical standpoint in the lab as well as in the eventual procurement of biological samples because these less toxic materials will provide the same phosphorylated enzymes in vitro or in vivo.

The OP analogs were used with hapten synthesis. The invention focused on the synthesis of enantiomerically pure OP analogs of five nerve agents they mimic.

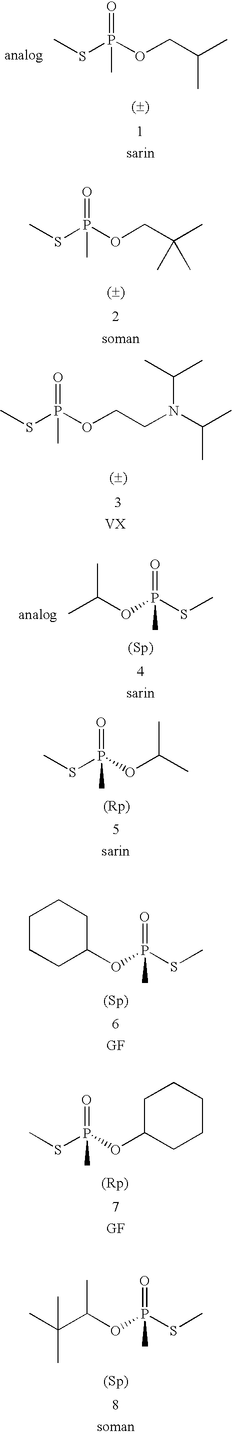

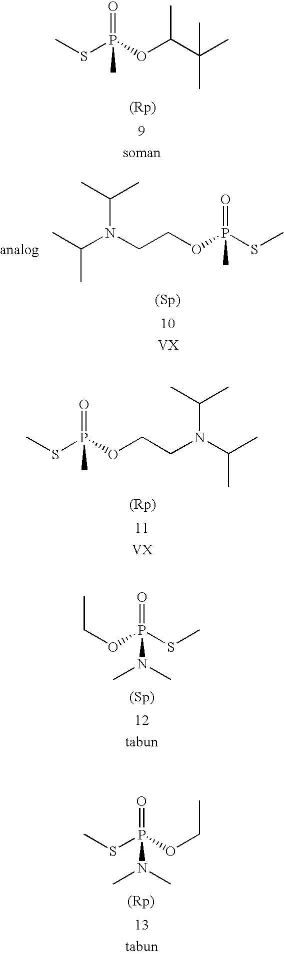

We have been able to synthesize and purify (>97%) both enantiomers of 4-13. The synthesis of the enantiomerically pure compounds 4-11 is shown in Schemes 14 and 15.

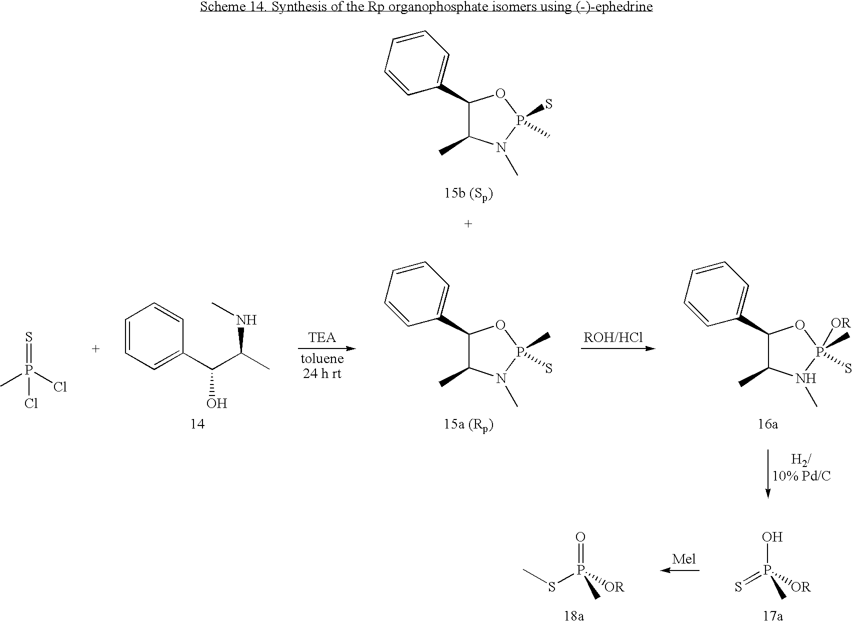

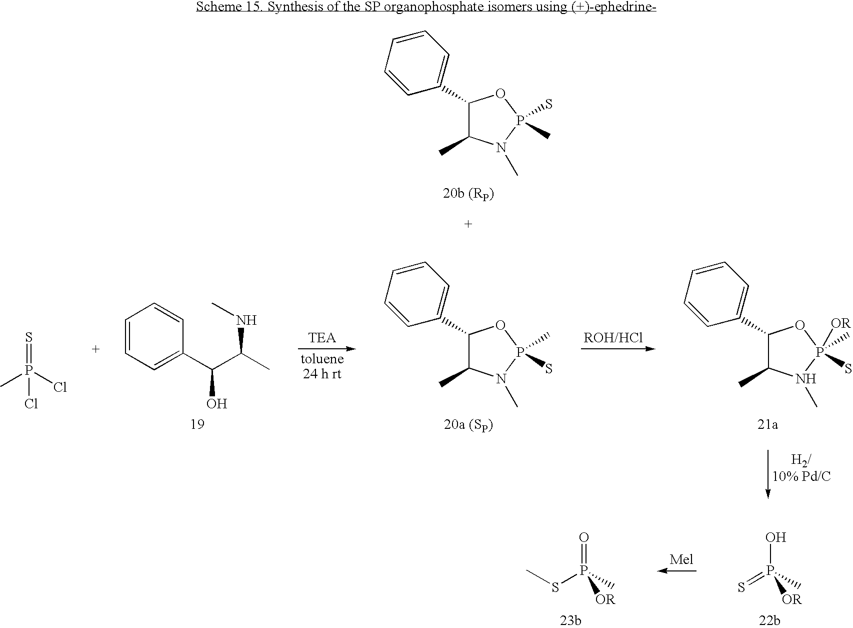

Since only one of the diastereomers of the oxazaphospholidinethione is formed over the other in the reaction of methylphosphonothioic dichloride with ephedrine, it is necessary to use both (+) and (−)-ephedrine to obtain high yields of each of the Rp and Sp oxazaphospholidinethione. The synthesis of compounds 12 and 13 uses a slight modification to the syntheses shown in Schemes 14 and 15 and are shown in Schemes 16 and 17.

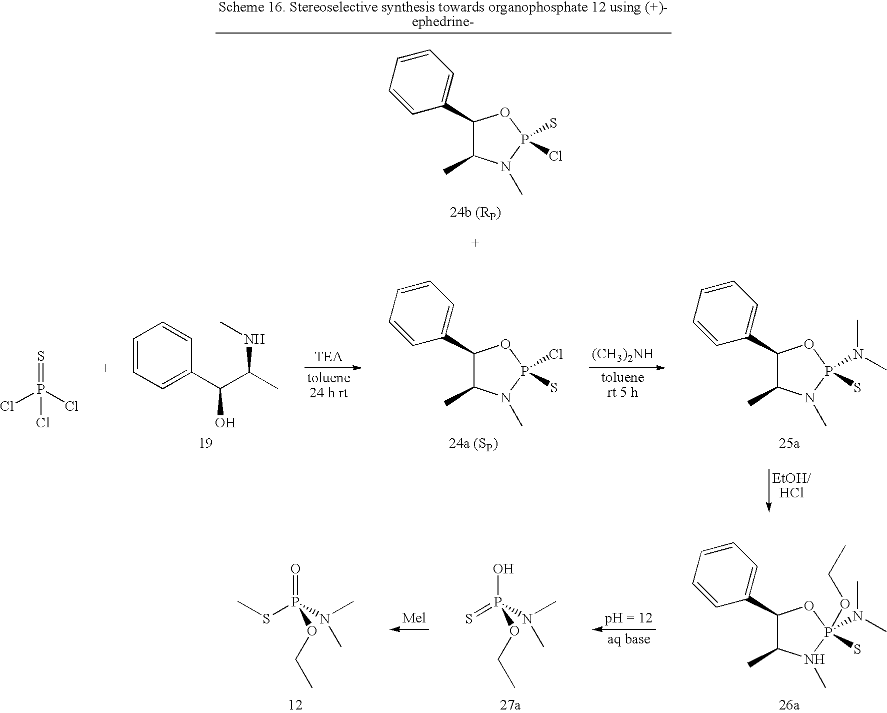

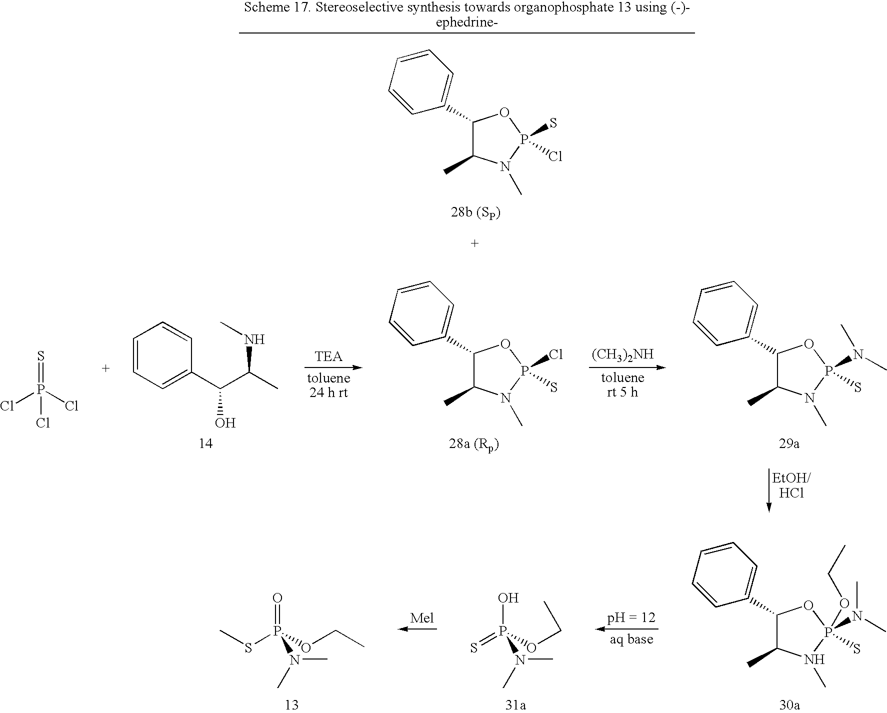

Synthesis of (2Rp,4S,5R) and (2Sp,4S,5R)-trimethyl-5-phenyl-1,3,2-oxazaphospholidine-2-thione (15a and 15b). A solution of 7.04 mL methylphosphonothioic dichloride (67.1 mmol) in 60 mL toluene was slowly added to a solution of 13.3 g (−)-ephedrine (80.5 mmol) in 46 mL triethylamine and 200 mL toluene t room temperature. The reaction mixture was covered and was stirred overnight at room temperature under argon and then filtered through Celite and the filtrate was washed with water and brine. The organic layer was dried over Na2SO4, filtered, and concentrated in vacuum. The crude product was purified by flash chromatography (EtOAc/hexane 1:5, 1:4) to yield 6.52 g (40%) of the predominant diastereomer 15a. The minor diastereomer 15b was isolated in much lower yield and usually as a mixture with 15a. 5a: white solid; 1H NMR (500 MHz, CDCl3) δ 0.75 (d, J=6.6 Hz, 3H), δ 2.06 (d, J=14.5 Hz, 3H), δ 2.77 (d, J=12.3 Hz, 3H), δ 3.64 (m, 1H), δ 5.66 (dd, J=6.1, 2.1 Hz, 1H), δ 7.27 (m, 2H), 7.31 (m, 1H), δ 7.35 (m, 2H). 15b: white solid; 1H NMR (500 MHz, CDCl3) δ 0.83 (d, J=6.5 Hz, 3H), δ 1.96 (d, J=14.2 Hz, 3H), δ 2.69 (d, J=12.7 Hz, 3H), δ 3.63 (m, 1H), δ 5.48 (dd, J=5.8, 3.3 Hz, 1H), δ 7.32 (m, 1H), 7.37 (m, 4H).

Synthesis of (2Sp,4R,5S) and (2Rp,4R,5S)-trimethyl-5-phenyl-1,3,2-oxazaphospholidine-2-thione (20a and 20b). The preparation is the same as described above but using (+)-ephedrine. 20a: white solid; 1H NMR (500 MHz, CDCl3) δ 0.75 (d, J=6.7 Hz, 3H), δ 2.06 (d, J=14.6 Hz, 3H), δ 2.77 (d, J=12.3 Hz, 3H), δ 3.64 (m, 1H), δ 5.67 (dd, J=6.0, 1.9 Hz, 1H), δ 7.28 (m, 2H), δ 7.32 (m, 1H), δ 7.37 (m, 2H). 20b: White solid; 1H NMR (500 MHz, CDCl3) δ 0.84 (d, J=6.5 Hz, 3H), δ 1.96 (d, J=14.2 Hz, 3H), δ 2.69 (d, J=12.7 Hz, 3H), δ 3.63 (m, 1H), δ 5.48 (dd, J=5.8, 3.3 Hz, 1H), δ 7.32 (m, 1H), 7.37 (m, 4H).

General preparation for the synthesis of Rp and Sp-O-alkyl S-methyl methylphosphonothioates. To a solution of 200 mg (0.83 mmol) of the appropriate oxazaphospholidine-2-thione, 2 mL of methyl ethyl ketone and 1.5 mL of the appropriate alcohol was added a solution of 0.7 mL of hydrogen chloride saturated alcohol in 0.7 mL methyl ethyl ketone slowly at 0° C. and warmed to room temperature. After stirring at room temperature for 2 h the reaction was quenched with 10 mL of ice-cold aqueous sodium carbonate (10%). The mixture was diluted with 7.5 mL water and 12 mL ethanol then set up for hydrogenation using 10% Pd/C (15 mg) and a balloon of H2. The system was covered, and the mixture was stirred overnight at room temperature. The catalyst was removed by filtration through celite, and the filtrate was concentrated in vacuo to remove the ethanol. The remaining aqueous layer was extracted extensively with ethyl ether, acidified with citric acid to pH 4, and extracted again with a mixture of isopropyl alcohol and chloroform (1:4). The second set of extractions was dried over Na2SO4, filtered, and concentrated to yield a yellow oil. The crude material was purified via preparative TLC. Spectral data for each isomer is identical.

(Sp)-O-isopropyl S-methyl methylphosphonothioate (4). See general preparation. Purification conditions 100% ether. Isolated 18 mg 1HNMR (500 MHz, CDCl3) δ1.32 (d, J=6.1 Hz, 3H), δ1.37 (d, J=6.1 Hz, 3H), δ1.75 (d, J=15.4 Hz, 3H), δ2.29 (d, J=12.9 Hz, 3H), δ 4.8 (m 1H).

(Rp)—O-isopropyl S-methyl methylphosphonothioate (5). See general preparation. Purification conditions 100% ether. Isolated 25 mg 1HNMR (500 MHz, CDCl3) δ1.32 (d, J=6.1 Hz, 3H), δ1.37 (d, J=6.1 Hz, 3H), δ1.75 (d, J=15.4 Hz, 3H), δ2.29 (d, J=12.9 Hz, 3H), δ4.8 (m 1H).

(Sp)-O-cyclohexyl S-methyl methylphosphonothioate (6). See general preparation. Purification conditions ether/hexane 4:1. Isolated 54 mg 1H NMR (500 MHz, CDCl3) δ1.23 (m, 1H), δ 1.36 (m, 2H), δ1.51-1.56 (m, 3H), δ1.72 (m, 2H), δ1.77 (d, J=15.6 Hz, 3H), δ1.92 (m, 1H), δ1.99 (m, 1H), δ2.30 (d, J=12.9 Hz, 3H), δ4.51 (m, 1H).

(Rp)-O-cyclohexyl S-methyl methylphosphonothioate (7). See general preparation. Purification conditions ether/hexane 4:1. Isolated 26 mg 1H NMR (500 MHz, CDCl3) δ1.23 (m, 1H), δ1.36 (m, 2H), δ1.51-1.56 (m, 3H), δ1.72 (m, 2H), δ1.77 (d, J=15.6 Hz, 3H), δ 1.92 (m, 1H), δ1.99 (m, 1H), δ2.30 (d, J=12.9 Hz, 3H), δ4.51 (m, 1H).

(Sp)-O-3,3-dimethyl-2-butyl S-methyl methylphosphonothioate (8). See general preparation. Purification conditions ether/dichloromethane 1:1. Isolated 27 mg 1H NMR (500 MHz, CDCl3) δ0.91 (s, 12H), δ1.36 (d, J=6.37 Hz, 3H), δ1.78 (d, J=15.6 Hz, 3H), δ2.33 (d, J=12.9 Hz, 3H), δ4.3 (m, 1H).

(Rp)-O-3,3-dimethyl-2-butyl S-methyl methylphosphonothioate (9). See general preparation. Purification conditions ether/dichloromethane 1:1. Isolated 12 mg 1H NMR (500 MHz, CDCl3) δ0.91 (s, 12H), δ1.36 (d, J=6.37 Hz, 3H), δ1.78 (d, J=15.6 Hz, 3H), δ2.33 (d, J=12.9 Hz, 3H), δ4.3 (m, 1H).

(Sp)-O-N,N-diisopropylaminoethyl S-methyl methylphosphonothioate (10). See general preparation. Purification conditions 100% ether. Isolated 9.6 mg 1H NMR (500 MHz, CDCl3) δ1.32 (d, J=6.2 Hz, 6H), δ1.38 (d, J=6.2 Hz, 6H), δ1.75 (d, J=15.4 Hz, 3H), δ2.30 (d, J=12.8 Hz, 3H), δ4.1-4.2 (m, 4H), δ4.8 (m, 2H).

(Rp)-O-N,N-diisopropylaminoethyl S-methyl methylphosphonothioate (11). See general preparation. Purification conditions 100% ether. Isolated 9.6 mg 1H NMR (500 MHz, CDCl3) δ1.32 (d, J=6.2 Hz, 6H), δ1.38 (d, J=6.2 Hz, 6H), δ1.75 (d, J=15.4 Hz, 3H), δ2.30 (d, J=12.8 Hz, 3H), δ4.1-4.2 (m, 4H), δ4.8 (m, 2H).

(2S,4R,5S)- and (2R,4R,5S)-2-chloro-3,4-dimethyl-5-phenyl-1,3,2oxazaphospholidine-2-thione (24a and 24b). To a solution of 6.9 g (41 mmol) of thiophosphoryl chloride in 25 mL toluene was slowly added to a slurry of 8.6 g (43 mmol) (+)-ephedrine, 35 mL of triethylamine in 150 mL of toluene and stirred at room temperature. After being stirred at room temperature overnight the reaction was poured into water and washed 3×150 mL of water. The organic phase was dried over sodium sulfate and concentrated in vacuo to give a yellow oil which solidified upon sitting. The crude material was purified via column chromatography (silica 9:1 hexane/ethyl acetate) to give 1.5 g of the Sp isomer and 2.5 g of a 95:5 mixture of the Sp and Rp isomers. 1HNMR (500 MHz, CDCl3) Sp isomer δ0.88 (d 3H), δ2.92 (d 3H), δ 3.85 (apparent dquint 1H), δ5.83 (d 1H), δ7.3-7.5 (m 5H). Rp isomer δ0.81 (d 3H), δ2.73 (d 3H), δ3.75 (apparent quintet 1H), δ5.6 (apparent triplet 1H), δ7.15-7.25 (m 5H).

(2S,4R,5S)-2-(N,N-dimethylamino)-3,4-dimethyl-5-phenyl-1,3,2oxazaphospholidine-2-thione (25a). To a solution of 500 mg of the 24a in 5 mL of dry toluene in a pressure tube, was bubbled anhydrous dimethylamine. After 1 min, the tube was sealed and stirred at room temperature. After 4 h, the reaction mixture was filtered, washed 2×5 mL of water, dried over sodium sulfate and dried in vacuo to afford quantitative 15. The crude material was used without further purification. 1HNMR (500 MHz, CDCl3) δ0.76 (d 3H), δ2.60 (d 3H), δ2.93 (d 6H), δ3.54 (apparent quintet 1H), δ5.67 (d 1H), δ7.27-7.37 (m 5H).

(Sp)-O-ethyl S-methyl N,N-dimethylphosphoramidothioate (12). To a solution of 500 mg (1.9 mmol) of 25a 2 mL of absolute ethanol was added a solution of 2 mL of hydrogen chloride saturated absolute ethanol. After stirring at room temperature for 2 h the reaction was basified to pH 12 with aqueous hydroxide and stirred at room temperature. After stirring overnight, the reaction mixture was extracted 3×20 mL ether and then excess methyl iodide (3 mL) was added and stirred an additional 1 h at room temperature. The reaction was diluted with water and extracted 4×20 mL of chloroform. The organic layers were combined and washed 3×15 mL water, dried over sodium sulfate and carefully concentrated under light vacuum. The crude material was purified via preparative TLC, 100% ether, to give a colorless oil. 1HNMR (500 MHz, CDCl3) δ1.34 (apparent t 3H), δ2.24 (d, J=14.2 Hz, 3H), δ2.74 (d, J=10.91 Hz, 6H), δ4.12 (m 2H).

(2R,4S,5R)- and (2S,4S,5R)-2-chloro-3,4-dimethyl-5-phenyl-1,3,2oxazaphospholidine-2-thione (28a and 28b). To a solution of 6.9 g (41 mmol) of thiophosphoryl chloride in 25 mL toluene was slowly added to a slurry of 8.6 g (43 mmol) (−)-ephedrine, 35 mL of triethylamine in 150 mL of toluene and stirred at room temperature. After being stirred at room temperature overnight the reaction was poured into water and washed 3×150 mL of water. The organic phase was dried over sodium sulfate and concentrated in vacuo to give a yellow oil which solidified upon sitting. The crude material was purified via column chromatography (silica 9:1 hexane/ethyl acetate) to give 200 mg of the Rp isomer and 1.5 g of a 95:5 mixture of the Rp and Sp isomers. 1HNMR (500 MHz, CDCl3) Rp isomer δ0.88 (d 3H), δ2.92 (d 3H), δ 3.85 (apparent dquint 1H), δ5.83 (d 1H), δ7.3-7.5 (m 5H). Sp isomer δ0.81 (d 3H), δ2.73 (d 3H), δ3.75 (apparent quintet 1H), δ5.6 (apparent triplet 1H), δ7.15-7.25 (m 5H).

(2R,4S,5R)-2-(N,N-dimethylamino)-3,4-dimethyl-5-phenyl-1,3,2oxazaphospholidine-2-thione (29a). To a solution of 200 mg of the 28a in 2 mL of dry toluene in a pressure tube, was bubbled anhydrous dimethylamine. After 1 min, the tube was sealed and stirred at room temperature. After 4 h, the reaction mixture was filtered, washed 2×5 mL of water, dried over sodium sulfate and dried in vacuo to afford quantitative 20a. The crude material was used without further purification. 1HNMR (500 MHz, CDCl3) δ 0.76 (d 3H), δ2.60 (d 3H), δ2.93 (d 6H), δ3.54 (apparent quintet 1H), δ5.67 (d 1H), δ7.27-7.37 (m 5H).

(Rp)-O-ethyl S-methyl N,N-dimethylphosphoramidothioate 13. To a solution of 200 mg (1.9 mmol) of 29a 2 mL of absolute ethanol was added a solution of 2 mL of hydrogen chloride saturated absolute ethanol. After stirring at room temperature for 2 h the reaction was basified to pH 12 with aqueous hydroxide and stirred at room temperature. After stirring overnight, the reaction mixture was extracted 3×20 mL ether and then excess methyl iodide (3 mL) was added and stirred an additional 1 h at room temperature. The reaction was diluted with water and extracted 4×20 mL of chloroform. The organic layers were combined and washed 3×15 mL water, dried over sodium sulfate and carefully concentrated under light vacuum. The crude material was purified via preparative TLC, 100% ether, to give a colorless oil. 1HNMR (500 MHz, CDCl3) δ1.34 (apparent t 3H), δ2.24 (d, J=14.2 Hz, 3H), δ2.74 (d, J=10.91 Hz, 6H), δ4.12 (m 2H).

Inhibition Kinetics of Synthetic Nerve Agent Analogs and Human Butyrylcholinesterase. The time-dependent kinetics of inhibition of human BuChE was examined with highly purified human BuChE as described in Example 8. The kinetic constants are listed below in Table 10.

TABLE 10
Kinetic constants for inhibition of huBchE.

| | kᵢ(× 10³ M⁻¹min⁻¹) | k₂ (min⁻¹) | K_{d} (μM) |
|---|---|---|---|
| Echothiophate | 1860 ± 135 | ND^{b} | ND |
| Cmpd 1 | 4.1 ± 0.12 | >0.8 | >200 |
| Cmpd 2 | 1.73 ± 0.42 | 0.62 ± 0.047 | 358.7 ± 85.7 |
| Cmpd 3 | 34.4 ± 0.42 | 1.04 ± 0.16 | 30.3 ± 11.8 |
| Cmpd 4 | 0.0668 ± 0.013 | 0.19 ± 0.014 | 2.89 ± 0.55^{c} |
| Cmpd 5 | 0.0145 ± 0.003 | 0.16 ± 0.020 | 11.42 ± 2.40^{c} |
| Cmpd 6 | 1.76 ± 0.20 | ND | ND |
| Cmpd 7 | 0.0137 ±0.003 | 0.031 ± 0.0023 | 2.29 ± 0.467^{c} |
| Cmpd 8 | 1.29 ± 0.1 | 0.53 ± 0.02 | 410 ± 30 |
| Cmpd 9 | 0.011 ± 0.003 | 0.008 ± 0.001 | 720 ± 210 |
| Cmpd 10 | 0.425 ± 0.118 | 0.587 ± 0.057 | 1380 ± 360 |
| Cmpd 11 | 1.24 ± 0.14 | 0.251 ± 0.003 | 202 ± 23 |
| Cmpd 12 | 0.032 ± 0.002 | ND | ND |
| Cmpd 13 | 0.011 ± 0 | ND | ND |

^{a}Highly purified human Butyrylcholinesterase;
^{b}ND, Not determined;
^{c}mM

As expected, the agents are significantly less potent inhibitors than echothiophate. It is notable that the Sp enantiomer of both the sarin analog and the GF analog is more potent an inhibitor of human BuChE than the Rp enantiomer. This is in agreement with the stereoselectivity of human BuChE inhibition by other organophosphates reported in the literature. This may be a useful feature of our overall strategy of obtaining agents that may have variable properties for in vitro and in vivo studies. Treatment of 1-13 (Table 10) with Che or albumin in vitro or in vivo will result in a phosphorylated protein or peptides that are used to procure antibodies to be used in the Array Biosensor.

### EXAMPLE 14

### Preparation and Testing of Antibodies to ChE-Organophosphosphonates of Nerve Agents

The acute toxic effects of OP compounds correlate well with their ability to inhibit ACHE by reaction with an essential serine hydroxyl to form a relatively stable phosphoserine ester bond. OP-ChE conjugates can serve as exquisitedly sensitive selective indicators of mechanism, information about adduct structure and potentially toxic outcomes. Likewise, phosphorylated albumin (i.e., Tyr 411) can serve as sensitive marker of OP or pesticide exposure. Antibodies have been developed and used to distinguish phosphonylated ChE or albumin by denaturing the enzyme to expose the active site to antibodies. Previously, antibodies have been used to probe ChE structure and allostery over catalytic activity. Selective antibodies are raised to specifically recognize individual OP-ChE or OP-albumin conjugates based on the precise modification imposed by the specific OP compound. This could be of enormous diagnostic value in ascertaining the relative toxic potential of an OP during or after an exposure. We chemically synthesized organophosphorylated serine or tyrosine octapeptides corresponding to the OP-Che or OP-albumin conjugate of nerve agents. With the haptens in hand, polyclonal or monoclonal antibodies are procured. The selective recognition of OP-ChE inactivated enzymes or OP-albumin from different species are examined. Antisera recognition is correlated with rates of enzyme inhibition, aging and oxime-induced reactivation. The antibodies are used in a panel of ChE biomarkers including phosphorylated albumin and other phosphorylated proteins for the selective classification of chemical exposures in biological samples. The antibodies are placed in the Array Biosensor to make a fieldable and efficient nerve agent OP and other OP-like material biosensor.

Synthesis of Haptens. The chemical synthesis of the requisite organophosphorylated serine or tyrosine octapeptides begins with selective esterification of Fmoc serine or tyrosine. Compounds 1-13 (Example 13) are prepared and used to treat with Fmoc serine on a preparative scale (100 mg) to afford the requisite protected organophosphorylated serine (Scheme 18).

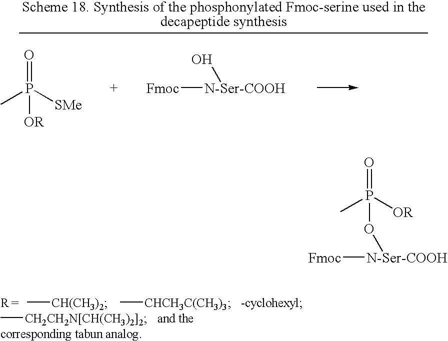

Nucleophilic displacement of S-alkyl methylphosphonothioates is done using the requisite sodium alkoxide, or bromine in alcohol and silver nitrate in alcohol. While the alkoxide and AgNO3/ROH reaction gave about 80% inversion of configuration at phosphorus, the displacement reaction using Br2/ROH afforded 100% inversion of configuration. The bromine-promoted substitution is usually fast and high yielding. However, it has also been reported that in certain sterically crowded situations, bromine promoted alcoholyses can take place with preponderant retention of configuration. Protection of the carboxylate may be necessary to achieve high yields. Alternatively, we will use the Br2/Fmoc-serine or -tyrosine reaction. If this reaction does not give satisfactory yields modifications will be employed. We have observed by NMR experiments that oxidative activation of 1-13 (Example 13) with meta chloroperbenzoic acid (MCPBA) forms an S-oxide that is rapidly attacked by nucleophiles. Thus, treatment of the S-oxide of 1-13 (Example 13) and addition of a trace of water rapidly quantitatively forms the corresponding alcohol (as judged by LCMS) where the S—(O)—CH3 group is replaced by an OH group. Treatment of 1-13 (Example 13) in the presence of 1.2 eq. MCPBA/CHCl2 for 5 mins at 4° C. followed by addition of 1 eq. FmocSer will also efficiently produce the desired OP-conjugated Fmoc serine or tyrosine. Incorporation of the OP-conjugated Fmoc serine or tyrosine into the normal octapeptide synthesis will provide the required OP-conjugated decapeptides used in the immunization studies.

Anti-Sera to the active site organophosphorylated serine of Che or Albumin. For human, primate and rat AChE and human, primate and rat BuChE, the five amino acids on either side of the serine active site are the same (i.e., TLFGESAGAAS) (SEQ ID NO:11). Based on the information, the decapeptide LFGESAGAAC (SEQ ID NO:12) is used to develop OP-conjugate-selective anti-sera. Partial sequence of Human Albumin (YKFQNALLVRYTKKVPQV) (SEQ ID NO:13), Rat Albumin (YGFQNAILVRYTQKAPQV) (SEQ ID NO:14) and Mouse Albumin (YGFQNAILVRYTQKAPQV) (SEQ ID NO:15) peptides are likewise be used for anti-sera procurement. Adducted protein can also be used directly. Of importance is the replacement of the terminal serine with a cysteine or a sulfur-containing linker. This will allow the necessary attachment chemistry to the biosensor (discussed below). The overall use of this decapeptide will conserve resources (as it will be of utility to both AChE and BuChE) and allow greater utility of the anti-sera. Below, ChE will stand for both enzymes (and by analogy, albumin). The 10S and 10SP designation refers to the non-phosphonylated and phosphonylated decapeptide. Anti-ChE10S and anti-ChE10SP anti-sera is generated by immunizing rabbits with ChE10S and ChE10SP peptides conjugated to keyhole limpet hemocyanin as described before. It is important to have anti-sera from both the OP-conjugated peptide (ChE10SP) and the native or non-conjugated peptide (ChE10S) to serve as a control. Conjugation is done by standard procedures. Rabbits are immuninized using standard procedures by a commercial lab. The requisite peptides are synthesized by standard procedures described above with the exception that the ChE10SP decapeptide will contain the same OP-conjugates arising from nerve agents 1-13 (Example 13). Anti-sera is purified by chromatography over a DEAE Affi-gel Blue column. The OP-conjugated decapeptides is coupled to Affi-gel 15 beads thru the Cys and the anti-sera is purified with these beads. Less specific anti-sera is eluted with 1 M NaSCN and decapeptide-specific fractions (eluted with 1 M glycine-HCl and immediately buffered to pH 8) is analyzed by ELISA to ensure specific antibodies. Western blots are done as above.

To characterize the specificity of the anti-sera, rat, primate and human AChE and BuChE or albumin is treated with 0.5 mM nerve agent analogs 1-13 (Example 13) or vehicle THF for 2 hr at 4° C. or until enzyme activity is decreased to 1-2% based on the Ellman colorimetric method. Enzyme is affinity purified from animal sera or from recombinant sources. Recombinant HuChE and primate BuChE are available in our lab and rat BuChE are purified from rat sera. AChE are purified from animal blood cell membranes. The inhibited enzymes are analyzed by immunoblot with a denaturing gel. As a positive control, immunoblots are probed with a polyclonal anti-ChE antiserum to show that equivalent recognition is present and the integrity of the enzyme is present. Competition experiments among the various OP-conjugated and non-conjugated enzymes are examined with the OP-selective anti-sera to show specificity. This will test our hypothesis that anti-ChE10SP is specific for the OP-conjugated serine and also specific for the type of conjugated OP. The effect of treatments are compared and samples from each treatment condition containing equal amounts of protein are examined within single gels and immunoblots. Densitometric analysis are used to quantify changes in the intensity of protein bands labeled with the anti-ChE10SP anti-sera and control anti-sera within the linear range for each antisera. The antisera are used in an Array biosensor to detect OP-derivatized Chef as a selective probe of OP exposure in biological fluids or environmental samples.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A method for detoxication comprising contacting a butyrylcholinesterase (BchE) with an organophospate agent, a drug of abuse, a gerbicide or a pesticide.

2. The method of claim 1, wherein the contact occurs in vivo.

3. The method of claim 1, wherein the contact occurs in vitro.

4. The method of claim 1, wherein the organophospate agent is a nerve agents selected from the group consisting of sarin, soman, GF, tabun, and VX.

5. The method of claim 1, wherein the organophospate agent is a compound selected from the group consisting of compound 1, 2, 3, 4, 5, 6, 7, 8, 9, 10, 11, 12 and 13.

6. The method of claim 1, wherein the drug of abuse is cocaine.

7. A method for screening a compound for butyrylcholinesterase (BchE) activity, the method comprising the steps of:
a) incubating the BchE with the compound; and
b) detecting the inhibition of BchE as an indication of biological or pharmacological activity of the compound.

8. The method of claim 7, wherein the BchE is rhesus monkey BchE or variants thereof.

9. The method of claim 7, wherein the compound is selected from the group consisting of compound 1, 2, 3, 4, 5, 6, 7, 8, 9, 10, 11, 12 and 13.

10. A method for making a mutation library of BchE in a vector.

11. The method of claim 10, wherein the vector is pENTRA or adenovirus vector.

12. The method of claim 10, wherein the BchE is rhesus monkey BchE or variants thereof.

13. The method of claim 10, wherein the BchE is human BchE or variants thereof.

14. The method of claim 10, wherein the BchE is a mixture of rhesus monkey BchE and human BchE or variants thereof.

15. A method for infecting a mammalian cell with a recombinant adenovirus by packaging of a mutation library of BchE in an adenovirus particle.

16. The method of claim 15, wherein the BchE is rhesus monkey BchE or variants thereof

17. The method of claim 15, wherein the BchE is human BchE or variants thereof.

18. The method of claim 15, wherein the BchE is a mixture of rhesus monkey BchE and human BchE or variants thereof.

19. A method for detecting BchE expression in culture medium of infected mammalian cells according to claim 9, comprising the steps of:
a) collecting a culture medium post infection; and
b) analyzing BchE activity in the culture medium.

20. The method of claim 19, wherein the BchE activity is analyzed using the Ellman method or western blot analysis.

21. The method of claim 19, wherein the BchE is rhesus monkey BchE or variants thereof.

22. The method of claim 19, wherein the BchE is human BchE or variants thereof.

23. The method of claim 19, wherein the BchE is a mixture of rhesus monkey BchE and human BchE or variants thereof.

24. A method for detecting BchE in prokaryotic or eukaryotic cells expressing BchE, the method comprising the steps of:
a) overlay the cells with agarose containing medium in the presence of an organophosphate compound selected from the group consisting of compound 1, 2, 3, 4, 5, 6, 7, 8, 9, 10, 11, 12 and 13;
b) incubation the mixture of step (a) for various length of time; and
c) detecting the BchE activity in overlay agarose containing buffer in the presence of butyrylthiocholine iodide and DTNB.

25. The method of claim 24, wherein the BchE is rhesus monkey BchE or variants thereof.

26. The method of claim 24, wherein the BchE is human BchE or variants thereof.

27. The method of claim 24, wherein the BchE is a mixture of rhesus monkey BchE and human BchE or variants thereof.

28. A method for detecting BchE by prokaryotic or eukaryotic cells expressing BchE, the method comprising the steps of:
a) overlay cells with agarose containing medium in the presence of a nerve agent or pesticide;
b) incubation the mixture of step (a) for various length of time; and
c) detecting the BchE activity in overlay agarose containing buffer containing butyrylthiocholine iodide and DTNB.

29. The method of claim 28, wherein the BchE is rhesus monkey BchE or variants thereof.

30. The method of claim 28, wherein the BchE is human BchE or variants thereof.

31. The method of claim 28, wherein the BchE is a mixture of rhesus monkey BchE and human BchE or variants thereof.

32. The method of claim 24 or 28, wherein the cell expression of BchE is derived from recombinant virus infection as described in claim 15.

33. A method of isolating recombinant virus expressing BchE, the method including the steps according to claim 24, further coring out agarose plugs from selectively stained yellow spot, and transferring the plugs to sterile medium.

34. A method for characterizing isolated plugs according to claim 33, further including the steps of infecting cells with medium incubated with plugs, and monitoring the BchE expression using the methods of any one of claim 19, 24 or 28.

35. A method of purifying individual recombinant virus from isolated plugs according to claim 33, the method including the steps of serial diluting the medium, infecting cells with diluted medium according to claim 15, detecting BchE according to claim 32, and isolating individual stained plugs according to claim 33.

36. A method of characterizing isolated plugs according to claim 33 or 35 by PCR amplification of BchE gene encoded by the recombinant virus from the culture medium incubated with the plugs, using adenovirus vector and/or BchE gene specific primers.

37. An isolated and purified DNA encoding a rhesus monkey butyrylcholinesterase (RhBchE) mutant and fragments thereof.

38. An array biosensor that is used in detection of an OP agent, a drug of abuse, a gerbicide or a pesticide.
